(19) United States
(12) Patent Application Publication
(10) Pub. No.: US 2015/0148258 A1
(43) Pub. Date: May 28, 2015

(54) METHOD FOR SIMULTANEOUS DETECTION, RECOVERY, IDENTIFICATION AND COUNTING OF MICROORGANISMS AND DEVICES FOR THE IMPLEMENTATION OF SAID METHOD

(71) Applicants: Claudio Rodriguez Martinez, Bejucal (CU); Jesus Eduardo Gonzalez Ruiz, Arroyo Naranjo (CU); Tamara Lobaina Rodriguez, Bejucal (CU); Raisa Zhurbenko, Bejucal Mayabeque (CU); Ana Iris Brito Gonzales, Bejucal Mayabeque (CU); Monica Lopez Hernandez, La Habana (CU); Javier Fernandez Aragon, La Havana (CU); Ivonne Alfonso Valdes, La Habana (CU); Adelaida Ortea Suris, La Habana (CU)

(72) Inventors: Claudio Rodriguez Martinez, Bejucal (CU); Jesus Eduardo Gonzalez Ruiz, Arroyo Naranjo (CU); Tamara Lobaina Rodriguez, Bejucal (CU); Raisa Zhurbenko, Bejucal Mayabeque (CU); Ana Iris Brito Gonzales, Bejucal Mayabeque (CU); Monica Lopez Hernandez, La Habana (CU); Javier Fernandez Aragon, La Havana (CU); Ivonne Alfonso Valdes, La Habana (CU); Adelaida Ortea Suris, La Habana (CU)

(21) Appl. No.: 14/389,568

(22) PCT Filed: Mar. 27, 2013
(86) PCT No.: PCT/CU2013/000002

§ 371 Date: Dec. 3, 2014

(30) Foreign Application Priority Data
Mar. 30, 2012 (CU) /P/2012/0055

(51) Int. Cl. C12Q 1/04; C12Q 1/14; C12Q 1/06; C12Q 1/10

(52) U.S. Cl. CPC C12Q 1/04; C12Q 1/10; C12Q 1/14; C12Q 1/06; G01N 2333/245; G01N 2333/21; G01N 2333/315; G01N 2333/40; G01N 2333/38
USPC 506/12; 506/37; 506/39; 506/40

(57) ABSTRACT

The present invention describes a method and devices for the simultaneous detection, recovery identification and counting of a plurality of microorganisms consisting in providing mixtures of nutrients specially selected from those that curtail the lag phase of growth in bacteria and moulds and which, together with fluorescent enzymatic, chromogenic or bioluminescent markers and other nutrient components or growth inhibitors, are embedded in three-dimensional structures or natural or artificial clays or ceramics with cavities of different dimensions and forms and specific surface areas of between 2×103 and 6×108 m²/m³.

This invention relates to the realm of microbiology—specifically, to the detection and identification of microorganisms in different environments and samples.

A significant number of conventional culture means and methods for detecting and identifying microorganisms are known, whose main disadvantages consist of an extended sample incubation period (at least 18 to 24 hours), along with cumbersome manipulation as it resorts to different means for isolating, enriching and identifying bacteria and yeasts.

Some solutions have been provided in order to reduce the identification time, such as the use of means with chromogenic and fluorogenic substrates, which are likewise too slow for identification needs in terms of availability.

The use of ceramic materials, including nanostructured ones, has been aimed mainly to concentrate microorganisms on samples for their further identification and for detecting or identifying monoclonal antibodies or fragments of genetic structures coupled to nanostructures (Integration of hydroxyapatite concentration of bacteria and seminested PCR to enhance detection of Salmonella typhimurium from ground beef and bovine carcass sponge samples. Elaine D. Berry and Gregory R. Siragusa. United States Department of Agriculture Agricultural Research Service 2. Roman L. Hruska U.S. Meat Animal Research CenterClay Center, NE 689330166. Accepted for Publication Feb. 15, 1999), (Method of manufacturing hydroxyapatite and uses therefore in delivery of nucleic acids. United States Patent Application US20080095820).

On the other hand, silicate nanoparticles have been used instead to inhibit the growth of microbes (Composition comprising aluminum silicates and silver nanoparticles as bactericides. WIPO patent application WO/2011/128488).

On U.S. Pat. No. 6,596,505 B2 from 2003, Ceri et al. proposed a device and methods for testing the effect of materials and surfaces in the formation of biofilms. The method comprises the use of hydroxyapatite and culture media for creating biofilms and the further identification of the characteristics of these microorganisms. The method does not consider identification in one single step, and requires attachments for forming biofilms, thus it is not quite suitable for identification purposes, since it is well known that the metabolic characteristics of microorganisms and their resistance to antimicrobial agents varies when biofilms are formed.

The invention of Hatzmann M. J. et al. (WO 2009/067012 A2) claims a method for detecting microorganisms on different liquid materials, and envisages the concentration of the microorganism on to filter-like device that is connected to other devices and whose filter is formed by to hydroxyapatite structure, to culture medium and chromogenic and fluorogenic substrates. The main limitations of the method are that a sample concentration phase is needed to detect contamination, that it only applies to liquid samples, that it needs further equipment, and that identification response is neither quick nor precise for many microorganisms as it is based on detecting glucuronidase or galactosidase activity.

In brief, the shortcomings of the methods described above on scientific bibliography and on patent documents consist of:
Not all microorganisms adhere to nanostructures (such as E. coli 0157:H7), and not all those that adhere can be recovered within a reasonable time for their future identification;
Adhesion of microorganisms depends on their concentration on the sample; the lower the concentration, the lower the adhesion;
In most methods it is necessary to separate microorganisms from structures for their future identification, which requires additional steps such as centrifugation, that in turn requires the use of additional equipment and exposes recovered microorganisms to ambient contamination or using centrifuges under asepsis conditions;
Those inventions that provide this additional phase require culture media in order to isolate microorganisms for their further identification through different immunoenzymatic methods or other molecular techniques;
The methods protected by the inventions described so far herein base their identification on detection mechanisms that resort to monoclonal antibodies that are very sensitive to temperatures and have a very short service life, or that use detection techniques for identifying DNA or RNA fragments that require additional equipment that may be unaffordable for small labs or labs that have limited resources;
Accelerated growth of microorganisms, therefore, under low concentrations, is very hard to identify and may not even be detected, or large volumes of sample must be filtered in order to detect these low concentrations;
In the few inventions that envisage microorganism contact with nano- or microstructures and with culture media, the latter are not chosen in particular due to their capacity of promoting quick growth, and that under low concentrations are not detected or whose detection occurs too late in regards to diagnostic needs;
Those nutritional bases existing in international markets that were chosen to be used on culture media are not able to promote the formation of enzymes as to multiply the biomass within a reduced period since they are not able to reduce the lag phase of microbial growth;
On similar structures based on nanoporous materials or formed by aggregated nanoparticles, to wide variety of microorganisms, such as yeasts, fungi, Gram-positive and Gram-negative bacteria, microbacteria and nanobacteria, are not detected at once;
Most patented methods require the sample being applied in liquid status, and in addition they must be concentrated by filtration, and they do not include the recovery of microorganisms suspended in air, other gases or in solid samples;
Some natural clays, such as zeolite, china clay/kaolin and others, because of their own mineral compound, become inhibitors of most microorganisms; therefore, they are not used for promoting growth, but rather its inhibition;
No method disclosed to date guarantees a simple, quick and simultaneous recovery, detection and identification of a variety of microorganisms that may be found on concentrations as low as 1 UFC/sample size and within a reduced time period.

The purpose of this invention consists in promoting a method for detecting, recovering, identifying and simultaneously enumerating a variety of microorganisms in different samples, as well as the devices required for its execution.

The novelty of this invention consists in the following:
The method envisages the intense and accelerated promotion and formation of cellular and enzyme structures through a combination of effects never described before—neither in scientific literature nor in patent documentation. These effects are basically achieved: a) through the use of nutritional compounds, specifically produced through original methods that reduce the lag growth phase and promote the growth acceleration phase; b) the acceleration of enzyme processes that degrade indicator substrates by contributing ions that may contain the three-dimensional structures of clays or ceramics; c) the mechanical effect from the adhesion of clay or ceramic structures that include combinations or cavities of different sizes that capture, retain or house microorganisms of the most different sizes, ranging from nanometers to colonies several centimeters long and filament structures of fungi, hyphae or spores and/or other propagules; d) the multiplication effect of the already increased contact surface provided by nano- and microstructures that significantly increase the enzyme decay speed on to solid phase; e) the effect of loads that may contribute ions over the surface of three-dimensional structures, including clay ones, or from the ingredients of nutritional compounds that may increase the adhesion of cells; and in general, contrary to all previous solutions, it is not based on the recovery, detection and identification of microorganisms merely on adhesion to structures (concentration);
This is the first time that clays or ceramics, such as zeolite, bentonite and kaolinite, among others, can be used in to natural way to exhibit bactericide, fungicide or bacteriostatic activity without requiring a chemical modification for promoting microbial growth, since this inhibiting effect caused by the natural presence on them of “toxic” ions for microorganisms is eliminated thanks to absorption or adsorption of nutritional mixtures designed especially for each kind of sample and three-dimensional structure. The prior applications of these clays have been aimed to eliminating bacteria;
There are no prior solutions that combine growth promotion over natural or artificial clays with the substrates, as on the prior solutions that employ chromogenic and fluorogenic substrates identification is carried out exclusively based on the action of said substrates in the presence of elevated cell concentrations, while on the other hand prior solutions used for promoting the growth of fungi or bacteria on clays or ceramics are not intended to identify, but instead for increasing the concentration of cells;
This is the first time that a variety of natural or artificial clays or ceramics are used in the same assay method or in an individual or composite device, each contributing different kinds of ions that work selectively as catalyzers of specific enzyme reactions of a species, genre or group of microorganisms which, along with the nutrients and growth factors contributed by the nutritional formulas chosen specifically for promoting a short period of the lag phase and for accelerating microbial growth, and which unexpectedly allowed detecting, recovering, identifying and/or enumerating a variety of microorganisms separately or inside the same sample within a period as brief as 60-90 minutes, which had never been achieved by chromogenic or fluorogenic methods of microbial identification;
Surprisingly, some microorganisms, such as specific species of Pseudomonas, exhibited characteristics that are not commonly exhibited on certain media; for example, when using siliceous earth with the nutritional compound Pseudomonas developed fluorescence after only 120 minutes, while on the rested culture media that contain this compound, fluorescence appear only after 18 hours;
It was unexpectedly detected for the first time that when cavity size is reduced, in particular that of three-dimensional structure pores of the tested devices, and thus when the contact surface and availability of surface loads was increased, the enzyme decay reaction of substrates accelerated and bacteria could be detected at least 60 minutes earlier when compared with similar structures containing the same nutritional compound and for the same microorganism, but with larger cavity sizes;
In method variants that envisage the use of three-dimensional structures with nutritional formulas, specific indicator substrates and antimicrobial agents on different parallel combinations as part of the aforementioned devices, it was possible to determine their sensitivity to antimicrobial agents along with the identification of microorganisms in a single step;
For the first time, hydrolyzed enzymes of Spirulina platensis algae, of Saccharomyces cerevisiae and of Torula; extract of sweet potatoes; extract of tomato; hydrolyzed papain of beef heart tissue and bovine blood; hydrolyzed enzymes of rennet whey lactoalbumin; hydrolyzed enzymes or casein buttermilk acids and hydrolyzed or autolyzed Eudrillus eugeniae with natural or artificial three-dimensional structures were combined for the first time in a device and as part of a method that unexpectedly shortened the period of the lag phase of microorganisms;
Sample contaminants, such as suspended solids in water that interfere with microbial identification and quantification could be eliminated on the same detection phase of the method;
It was possible to achieve an extremely versatile device that, in the form of single or multiple combined units, allows carrying out the detection, recuperation, identification and/or recount of the most varied microorganisms on different kinds of samples, from gaseous, liquid, solid, gels and zoles with contamination levels of less than 1 UFC/sample unit, up to 10⁹ UFC/sample unit, with no contaminant interference from samples.

The advantages of this method and of the device consist of:
All viable microorganisms may be detected, recovered, identified and/or enumerated at once, since the principles of the method guarantee all conditions required for this purpose;
The proposed method and devices are able to detect and/or recover all microorganisms, whether they adhere to the structure or not, unlike prior solutions to this invention that do not guarantee that all microorganisms will adhere to the nanostructures (such as E. coli 0157:H7), nor that all those adhered may be recovered within a reasonable time for their future identification;
Since this method is not based merely on the adhesion of viable microorganisms to the structures of the device, all microorganisms present on the sample are detected, even under low concentrations or from their spores, hyphae or other propagules, and this is achieved unlike the previous methods, in which the adhesion of microorganisms depends on their concentration on the sample (the lower the concentration, the lower the adhesion);
The method envisaged in this invention allows detecting, recovering, identifying and/or enumerating, regardless of the original characteristics of the structures, their ions or their pH, unlike some of the aforementioned patents on which the adhesion of microorganisms to their structures for their concentration and subsequent identification is carried out through the ionic activity over the surface of those structures that interact with the microbial cells, in such a way that if the sample carrying the microorganisms contains substances that may interfere or seize these ions, or if their pH affects them, the correct and timely detection, identification or quantification may not be achieved;
The proposed invention does not require any secondary identification steps, sample concentration, special asepsis conditions or mandatory equipment, so it differentiates itself from previous descriptions because most of them need to separate microorganisms from the structures for their further identification, which includes additional steps such as centrifugation, which requires additional equipment (centrifuges) and exposes the recovered microorganisms to ambient contamination or to using centrifuges under asepsis conditions;
The new method is simple, low cost, identification does not take long and does not require additional technological techniques—unlike other disclosed inventions that envisage one or more additional phases, require culture media separate from the structures in order to isolate the microorganisms for their subsequent identification by different methods, including immunoenzymatic methods or other molecular techniques of identification and make it more expensive and technically complex;
The method described on the patent application is carried out with safety and under different lab and ambient conditions, and is highly stable since the reagents, preparations and nutritional compounds, as soon as they are embedded on the three-dimensional structures of the devices are very stable under the ambient temperature and humidity of the lab, which represents significant differences between the methods and devices previously mentioned on the scientific and patent bibliography, since in general identification is based on detection mechanisms with monoclonal antibodies that are highly sensitive to temperatures or use detection techniques for locating DNA or RNA acid fragments that require additional equipment that are too expensive for small labs or labs with scarce resources;
The execution of the method is very stable, so it allows carrying out each step with extended periods between them on the required cases since, as it was previously mentioned, when the nutritional formulas are absorbed by the three-dimensional structures with nano- and microcavities and after the solvent has been eliminated, the devices are very stable before temperature and humidity changes, and therefore can be used safely over long periods. This effect is due to the fact that the solids of nutritional formulations have very low humidity and the residual humidity is subject to adsorption forces from cavity surfaces that form the device and thus are not available for biological or biochemical decay reactions, until the second solvent or the sample is added;
This invention describes a method with a very low detection limit per sample unit of the nutritional compounds used (lower than 1 UFC), which means that microbial species may be detected and recovered under very low concentrations, thus facilitating their identification and/or enumeration, and unlike other state-of-the-art procedures disclosed, it does not require large sample volumes or high inoculation concentrations;
Low concentrations (less than 1 UFC/sample unit) of microorganisms from different species, genres, groups and sizes are detected at once by this method thanks to contact with those nutrients and market substrates specifically selected for different three-dimensional structures with different characteristics; this is carried out differently from the concepts of other authors by the fact that those nutrients are not chosen specifically for their capacity of promptly promoting growth under specific conditions of the structures and characteristics of the microorganisms to be detected and thus, under low concentrations, some of them are not detected or their detection takes too long in regards to diagnostic needs;
In the same structure or device, to wide variety of microorganisms, such as yeasts, filamentous fungi, positive-Gram and negative-Gram bacteria, microbacteria and nanobacteria are detected in unison; this effect had not been achieved before by other procedures;
The invention described in this document allows, along with the same combination of structure-nutritional compound enzymatic markers, detecting, recovering, identifying and/or enumerating those microorganisms suspended in gases, for example in air, liquids or solid samples, which are steeply differentiated from traditional procedures that use other tools, supports, components and different culture media depending on the type of sample to be treated, as in the case of liquid analysis samples in which special media and filtration membranes made from acetate or cellulose nitrate are used; however, for solid samples, these membranes are not used as these media have variations in their formula or even the plate size is different;
According to this invention, all kinds of natural clays, ceramics and calcium phosphates can be used with no constraints, since they are combined on the devices with the nutritional compounds and other components that neutralize the inhibitor effect that may be caused by some of the ions they contain, such as zeolite, kaolinite or bentonite, just by mentioning three examples; this represents a major difference with the use of these clays envisaged on prior solutions, that intend to use them as antibacterial agents;
For the purposes of this method (detecting, recovering, identifying and/or enumerating), in order to implement the method and configure the devices, natural clays which originally exhibited bactericide, fungicide or bacteriostatic activities may be used, sparing additional purification or chemical modification expenses for promoting microbial growth, thanks to their combination with the other components described on the previous paragraph;
The method allows determining the sensibility or resistance to antimicrobial agents along with their identification within a very brief period for any microorganism species, genre or group that is present in the sample, thanks to the combination of different three-dimensional structures with different nutritional compounds, enzymatic markers with the selected antimicrobial agents;
The new device is simple, low cost, and can be easily prepared for its manufacture.

A detailed description of the invention is given below.

The nutritional components of this invention are chosen among a series of mixtures of proteins, carbohydrates, vitamins and minerals that are degraded through chemical or enzyme methods. One or more of these nutritional mixtures that stimulate microbial growth are prepared in aqueous solutions or salt solutions with concentrations of 0.1 to 3 g/L that are inoculated with 0.1 ml of the microorganisms intended to be detected, recovered, identified or enumerated under the concentration of 3×10⁸ UFC/ml and which are incubated at the desired temperature and oxygen tension, measuring the microbial growth kinetics through any known method, preferably determining the increase of optical density over time. Those compounds that ensure a reduction of the lag growth phase, which does not surpass 60-120 minutes for bacteria and 16 hours for yeasts and filamentous fungi, are selected.

Some examples of the nutritional components mentioned above are hydrolyzed enzymes of Spirulina platensis algae described in Cuban Invention Copyright Certificate No. 22310; extract of Saccharomyces cerevisiae obtained through enzyme hydrolysis as described in Cuban Invention Copyright Certificate No. 22221, and hydrolyzed enzymes of Torula fodder yeast (Cuban Invention Copyright Certificate No. 22280); extract of sweet potatoes, as disclosed in Cuban patent No. 23507; tomato extract (Cuban Invention Copyright Certificate No. 22308); hydrolyzed enzymes from beef heart tissue (Cuban Invention Copyright Certificate No. 22442), from bovine blood (Cuban Invention Copyright Certificate No. 22208) and from beef liver (Cuban Invention Copyright Certificate No. 22220); hydrolyzed enzymes of lactoalbumin from rennet whey (Cuban Invention Copyright Certificate No. 22219), hydrolyzed enzymes or casein acids from buttermilk (Cuban Invention Copyright Certificates No. 22166 and 22089, respectively) and hydrolyzed or autolyzed Eudrillus eugeniae (Cuban Invention Copyright Certificate No. 22381). The selected compounds are dissolved or suspended in a first solvent in amounts ranging from 1 to 50 g/L.

To all of the above there can also be added further hydrolyzed enzymes, hydrolyzed chemicals, such as peptones and triptones or commercial protein extracts from algae, microorganisms, vegetables, higher animal tissue and their combinations, such as those obtained from beef meat, brains and potatoes, among others, in quantities ranging from 1 to 10 g/L.

As soon as the compound is prepared, one or more chromogenic, fluorogenic or bioluminescent enzymatic markers compounds in quantities ranging from 0.01 to 2 g/L can be added. Some examples of these markers may be: compounds derived from phenol, such as ortho- and paranitrophenols, paranitroaniline, indolyl derivatives: 5-bromo-4-chloro-3-indolyl, 5-bromo-6-chloro-3-indolyl (magenta), 6-chloro-3-indolyl (salmon), derivatives of methylcoumarin and methylumbelliferyl (MUG) for detecting activity of galactosidase, glucuronidase, decarboxylase, glycosidase and phosphatase, among others.

Other substances can be added to the mixture of nutritional compounds and enzymatic markers, such as microorganism promoters or inhibitors belonging to certain genres, species or groups. Some examples of these substances include vitamins, mineral salts, albumin, antibiotics, dyes, tints, bile salts, beef bile, sugars and amino acids. Likewise, other substances that increase the solubility of enzymatic markers or the permeability of microorganism cells can be added in quantities ranging from 0.01 up to 40 g/L.

If it were intended to determine the sensibility to antimicrobial and antifungal agents or to cleansing or disinfecting solutions, or for proving the particular bactericide or bacteriostatic effect of some substance or product, salts, resins, natural plant extracts, fatty acids, esters, bactericides, bacteriostatics, alcohols, substances with superficial activity or their mixtures in quantities ranging from 0.01 to 2 g/L and/or antibiotics or antifungal agents in quantities ranging from 10 to 100 μg/L may be also added to the nutritional compound selected.

The nutritional compound selected, along with enzymatic markers and other components, is dissolved or dispersed in a first solvent in quantities ranging from 1 to 150 g/L.

The solvent may be distilled or deionised water, aqueous salt solutions (NaCl, phosphate solutions among others), alcohols and alcohol solutions (e.g., 10% p/v basic fuchsine solution in ethyl alcohol), solutions of substances that increase the solubility of enzymatic markers [e.g., dimethyl sulfoxide (DMSO)] or the permeability of microorganisms cells.

Once the nutritional mixture is formed and dissolved or suspended in the first solvent along with the enzymatic markers and other components, they can be sterilized through any known method, except those compounds containing thermolabile substances that cannot be sterilized through heat.

Once the nutritional mixture is formed and dissolved or suspended in the first solvent along with the enzymatic markers and other components, they may come into contact with one or more three-dimensional structures of natural or artificial clays or ceramics.

These three-dimensional structures may be previously sterilized through any known method.

The contact time of the nutritional compound and other components dissolved or suspended in the first solvent and the three-dimensional structure of clay or ceramic ranges in general, from 10 minutes for nanometric or submicrometric (<1000 nm) dimensions, up to 60 minutes for larger structures.

These structures must have a specific surface of 2×10³ to 6×10⁸ m²/m³, and be formed by to variety of nano-, micro- and macro-cavities or their combinations.

These clays and/or natural or artificial ceramics are chosen from kaolinite, halloysite, dickite, nacrite, chrysolite, antigorite, lizardite, vermiculite, mica, hectorite, saponite, hydrotalcite, muscovite, chlorite, diatomaceous earth, bentonites (montmorillonite, sauconite, beidelenite, nontronite) clinoptilotites, hydroxyapatites, zeolites and calcium phosphates or their combinations.

The calcium phosphate structures mentioned in the paragraph above must be chosen between: metaphosphate [Ca(PO₃)₂], monohydrated monocalcium phosphate [Ca(H₂PO₄)₂H₂O], dihydrogen phosphate tetracalcium (Ca₄H₂P₆O₂₀), heptacalcium phosphate [Ca₇(P₅O₁₆)₂], calcium pyrophosphate (Ca₂P₂O₇ and Ca₂P₂O₇2H₂O), dicalcium phosphate [CaHPO₄, CaHPO₄.2H₂O and Ca(H₂PO₄)₂], tricalcium [Ca₃(PO₄)₂], octacalcium phosphate [Ca₈H₂(PO₄)₆.5H₂O], calcium-deprived hydroxyapatite [Ca₁₀-x(HPO₄)x(PO₄)₆-x(OH)₂-x], hydroxyapatite [Ca₁₀(PO₄)₆(OH)₂], tetracalcium phosphate [Ca₄(PO₄)₂], apatite [Ca₁₀(PO₄)₆(OH,F,Cl,Br)₂], carbonate apatite [Ca₅(PO₄,CO₃)₃(OH,F)] or a mixture of them. Clays and/or natural or artificial ceramics and calcium phosphates may have isomorphic ion replacements with cations or previously functionalized with different ions, preferably monovalent, divalent, trivalent or tetravalent, acting as enzyme catalyzers such as Na, K, Ca, Mg, P, Fe and Zn, forming superficial layers or distributed throughout its entire structure.

The three-dimensional structures mentioned above are selected between those whose cavity dimensions correspond to:
nanocavities or particles, preferably with shrivelled surfaces, with diameters or clearances up to 200 nm for nano- and microbacteria;
nano- and submicrocavities with diameters or clearances from 5 nm to 1000 nm for different sized bacteria;
microcavities with diameters or clearances from 1 μm to 1000 μm for yeast bacteria and cells;
micro- and macrocavities with diameters or clearances of more than 1 μm and up to 2 mm for bacteria, yeasts and filamentous fungi;
combinations of all cavity diameters and clearances, from nano- up to macro- of 2 mm for the microorganism assortment.

Cavities on the structure may appear as pores, channels, tubes, regular or irregular bags of different geometric shapes or their combinations, or if available as layers or sheets.

Three-dimensional structures may form a 5 nm to 1 mm thick layer or film, in particular when used for detection, identification or enumeration of microorganisms using the membrane filtration technique or for detection superficial microorganisms; or a column up to 10 cm high, in particular when it is required to filter large quantities of liquid microorganisms; suspensions that may be found with low concentrations, or when different structure zones are used with different enzymatic markers or with different nutritional mixtures.

Structures may also be used as spheres or pearls with a diameter of 5 nm to 10 mm; hexagons or cubes forming a set of test with different compounds, or they may be added to liquid or suspension sample containing the microorganisms.

Other structures shaped as cylinders or tubes with a very small diameter (5 nm) for small aliquots that join many of these tubes to form a set with a diameter of up to 10 cm, resembling the diameter of a Petri dish for those tests provided by recount standards that require such a surface.

The height of these structures varies depending on the presentation format to use on the method, ranging from 5 nm for nanoaliquots, or microaliquots, or for joining many of these structures in a sandwich-like set of layers that may reach up to 10 cm high.

Three-dimensional structures may appear as fibres or webs that hold the microorganisms, allowing their detection.

Whenever it is desired that structures detect and identify microorganisms throughout the entire volume of a sample, clays are used that are able to swell a lot naturally or by adding jellifying substances that make them adopt the shape of the container holding them.

Three-dimensional structures according to this invention may exhibit multiple zones with different porosities, diameters or clearances of nano-, micro- and macro-cavities throughout their volume, length or diameter, distributing these zones as a gradient or in differentiated zones. Different nutritional compounds and enzymatic markers may be added in each zone, throughout their structure, length or diameter, thus distributing over concentric zones. Distribution of compounds or the concentration of one or more of their components may be ensured through continuous or discontinuous gradients.

To these structures substances may be added that contribute to fixing the nutritional compound and the selected enzymatic markers and other components in those cases in which it is suspected that the sample fluid may drag said compound. Some of these substances can include alginates, such as sodium; natural polysaccharides, such as pectin and quinine; gum Arabic and other gums; starches, such as corn or yam starch, or pre-gelatinized starches; dextran and carboxymethylcellulose and other derivative polymers; carrageenan or sodium carrageenate; agar; agarose and artificial polymer derivatives; vinyl alcohol derivatives, as well as polybutylene and polypropylene; and polyvinylpyrrolidone of different molecular weights in quantities ranging from 0.01 to 0.5 g/g with three-dimensional structure.

In the event that the specific surface of a three-dimensional structure is lower than 500 m²/g, substances may be added that increase its absorption capacity, such as activated carbon and cellulose in quantities ranging from 2 to 4 mg/g, for example, as layers.

When the absorption is completed, the first solvent is eliminated. The most recommended procedure is under ambient temperature and atmospheric pressure with forced air circulation in order to preserve nutrients and prevent their decay, as in the case of thermolabile vitamins.

The first solvent may be eliminated by drying the three-dimensional structure at a temperature between 25 and 110° C. under atmospheric pressure or under pressure lower than the atmosphere, such as a vacuum oven between 30 minutes and 3 hours, or eliminating it through sublimation or aspersion drying at a temperature of 90 to 180° C.

After the first solvent has been eliminated, the structure may be preserved up to the moment when the assays are conducted for periods of up to five (5) years. It is advisable to sterilize these structures with the compounds embedded, preferably but not exclusively by other means of radiation. Other methods may be used, such as autoclave sterilization for those structures that do not contain any thermolabile components.

The capacity of recovering target microorganisms is proven by selecting those structures that have a detection limit of less than 1 UFC/10 L for liquid samples, less than 1 UFC/250 g for solid samples or less than 1 UFC/10 m³ of air and a maximum limit of up to 10⁹ UFC/ml or 10⁹ UFC/g or 10³ UFC/m³.

Prior to staring the assay, the three-dimensional structure may be placed over supports shaped as plates, layers or cylinders that are pervious to gases or liquids or impervious to them; or surrounded by impervious materials on at least 90% of its surface.

Later on, those microbial cells that may be formed by a variety of microorganisms may be placed in contact between them in order to be detected, recovered, identified and/or enumerated among a species, genre, group or combinations of them, including nanobacteria, bacteria, moulds and yeasts, as well as spores, hyphae or other propagules with three-dimensional structures in the presence of a second solvent.

This second solvent may be water, a hypotonic, isotonic or hypertonic solution of salts, such as sodium chloride, or the sample itself depending on the nature of those microorganisms to be identified.

Some examples may include biological samples such as blood or food, like milk or sample suspensions.

Another variant of the method consists in placing a microorganism suspension in contact with gaseous carriers such as air or aerosols.

The sample is applied to the structure on the following ratios: 0.05 to 13 ml/g, or from 0.1 to 10 m³/g of the three-dimensional structure.

In order to detect, recover, identify or quantify the diversity of cells, the second solvent or the samples holding them may be placed in contact with the surface of the three-dimensional structure or making it pass through it, down to a certain depth; if cells or the samples that hold them are in the form of a suspension in gas or liquid phase, as a gel or with semi-solid or solid consistency, applying it directly over the structure, or through an application device such as, for example, a swab, a holder or a needle, among others.

The samples or the second solvent holding the microorganisms may be applied over different structures at the same time.

The three-dimensional structure is then subject to temperatures between 20 and 50° C. over a period longer than the greater duration of the lag phase or of the final growth acceleration phase of the microorganism with the slowest development, under variable oxygen tension ranging from aerobic conditions to the total absence of this element, depending on the target microorganisms to be detected.

Growth will be observed from 30 minutes up to 240 minutes for bacteria, and along with detection different species, genres or groups may be identified. For this purpose, structures are maintained under the aforementioned conditions in order to foster microbial growth both inside the cavities and on the surface.

For those microorganisms of very slow growth, such as yeasts and filamentous fungi, growth is observed in only 16 hours, instead of 36-72 hours observed in traditional methods.

Growth is observed on nanoparticles or cavities through nanobacterial activity, and that of other bacteria is observed indirectly over the products from the breakup of enzymatic markers that, under the action of microbial enzymes, may accumulate in them. Smaller-sized bacteria develop in microcavities, while larger bacteria, yeasts and filamentous fungi grow on macrocavities and all other microorganisms grow over the surface.

The detection and identification of the variety of cells is basically, but not exclusively, carried out through visual or automatic detection of fluorescence or bioluminescence.

Further methods may be used, such as: for the change of the three-dimensional structure or of its color, consistency, texture, shine, opacity, tone, uniformity or transparency; or through changes of color, shine, tone, transparency or fluorescence of the second solvent or of the sample; or by the appearance of bioluminescence, both inside the cavities and over the surface of the structure; or by observing other morphologic structures; or through metabolic reactions on the three-dimensional structure, in the second solvent or in the sample; or through a combination of some or all identification means.

Cell concentration on the sample is determined over the surface of the structural layers or coats, through visual enumeration, through automatic superficial methods, or by measuring the intensity of the fluorogenic colorimetric or bioluminescent signal under ultraviolet, visible or infrared light, of electrical, thermal or magnetic signals, through pH changes or by quantifying the emission or consumption of gases produced by the activity of microorganisms during the lag phase or the growth acceleration period, such as carbon dioxide, oxygen, hydrogen sulphur, ammonia and hydrogen. Along with the identification of the variety of microorganisms, the resistance or sensibility to antimicrobial agents such as bactericides, bacteriostatics, fungicides, cleaning solutions, added to the mixture of nutritional compound and enzymatic markers may be determined, observing total or partial growth inhibition, its deceleration, the extension of the lag phase, or due to the absence of substrate decay reaction.

The method may be carried out with the aid of devices formed by a nutritional mixture that fosters microbial growth, selected among the hydrolyzed enzymes of alga Spirulina platensis; hydrolyzed enzymes of Saccharomyces cerevisiae and of Torula; extract of sweet potatoes; tomato extract; hydrolyzed enzymes of beef heart and liver tissue and of bovine blood; hydrolyzed enzymes of lactoalbumin from rennet whey, hydrolyzed enzymes or casein acids from buttermilk and hydrolyzed or autolyzed from Eudrillus eugeniae and its combinations and one or multiple chromogenic, fluorogenic or bioluminescent enzymatic markers absorbed and/or adsorbed on a three-dimensional structure of clay or natural or artificial ceramics, selected from kaolinite, halloysite, dickite, nacrite, chrysolite, antigorite, lizardite, vermiculite, mica, hectorite, saponite, hydrotalcite, muscovite, chlorite, diatomaceous earth, bentonites (montmorillonite, sauconite, beidelenite, nontronite) and calcium phosphates or their combinations formed by a variety of nanocavities or particles; submicro-, micro- and macrocavities.

Devices according to this invention contain all the components required for implementing the method, whereas those components are available dehydrated on quantities that ensure the concentrations of each of them as described in the original method.

These devices may be formed by calcium phosphates chosen among: metaphosphate [Ca(PO₃)₂], monohydrated monocalcium phosphate [Ca(H₂PO₄)₂H₂O], dihydrogen phosphate tetracalcium (Ca₄H₂P₆O₂₀), heptacalcium phosphate [Ca₇(P₅O₁₆)₂], calcium pyrophosphate (Ca₂P₂O₇ and Ca₂P₂O₇2H₂O), dicalcium phosphate [CaHPO₄, CaHPO₄.2H₂O and Ca(H₂PO₄)₂], tricaicium [Ca₃(PO₄)₂], octacalcium phosphate [Ca₈H₂(PO₄)₆.5H₂O], calcium-deprived hydroxyapatite [Ca₁₀-x(HPO₄)x(PO₄)₆-x(OH)₂-x], hydroxyapatite [Ca₁₀(PO₄)₆(OH)₂], phosphate tetracalcium [Ca₄O(PO₄)₂], apatite [Ca₁₀(PO₄)₆(OH,F,Cl,Br)₂], carbonate apatite [Ca₅(PO₄,CO₃)₃(OH,F)] or a mixture of them. They may also contain mixtures of the substances mentioned with the phosphates described in the previous paragraph.

Some of these devices may be formed by natural or artificial clays, ceramics and other calcium phosphates with isomorphic ion replacements with cations or be previously functionalized with different ions or monovalent, divalent, trivalent or tetravalent cations, forming superficial layers or distributed throughout their entire structure. These cations may be Na, K, Ca, Mg, P, Fe and Zn that essentially play the role of catalyzers on the marker decay enzyme reaction.

Three-dimensional structures of these devices exhibit cavities in the form of pores, channels, regular or irregular sacks with different geometric shapes or their combinations; or they are available as plates or layers, depending on the type of clay or ceramic used and based on their production technology, these cavities are classified depending on how they will be used in the method as follows:
nanocavities or particles, preferably of shrivelled surfaces, with diameters or clearances of up to 200 nm for nano- and microbacteria;
nano- and submicrocavities with diameters or clearances of 5 nm to 1000 nm for bacteria of different sizes;
microcavities with diameters or clearances of 1 μm to 1000 μm for yeast bacteria and cells;
micro- and macrocavities with diameters or clearances of more than 1 μm and up to 2 mm for bacteria, yeasts and filamentous fungi;
combinations with all diameters or clearances of cavities from nano- up to macro, up to 2 mm for the entire variety of microorganisms.

These devices contain one or more nutritional compounds, whose components are chosen among mixtures of proteins, carbohydrates, vitamins and minerals decayed by chemical or enzyme methods that ensure that the lag phase does not exceed 60-120 minutes for bacteria and 16 hours for yeasts and filamentous fungi in quantities ranging from 0.33 to 20 mg/g of three-dimensional structure.

The devices also contain one or multiple chromogenic, fluorogenic or bioluminescent enzymatic markers inside the cavities or over the surface of the structures in quantities ranging from 0.0033 to 0.66 mg/g of three-dimensional structure. In addition to these substrates, the devices may contain pH and potential redox indicators.

In addition, the nutritional compound inside the device may include further commercial products such as hydrolyzed enzymes, hydrolyzed chemicals or algae protein extracts, microorganisms, vegetable components, higher animal tissue and their combinations in quantities ranging from 0.33 to 4 mg/g of three-dimensional structure. Some examples of these substances include bacteriological peptone, triptone, meat, brain and heart extracts, potato, corn, rice, soy and yeast extracts.

The devices may include other substances such as growth promoters, inhibitors, salts, buffers, carbohydrates and other components used for promoting the growth of those microorganisms belonging to certain genres, species or groups in quantities ranging from 0.003 up to 14 mg/g.

Overall, those components located inside the cavities or over the surface of the devices (mixture of the nutritional compound with enzymatic markers and other components) are found in quantities ranging from 0.33 to 60 mg/g of the three-dimensional structure.

Devices according to this invention have detection limits of less than 1 UFC/10 L for liquid samples, less than 1 UFC/250 g for solid samples or less than 1 UFC/10 m³ and a maximum limit of up to 10⁹ UFC/ml or 10⁹ UFC/g or 10³ UFC/m³.

Those devices used for selectively detecting, recovering, identifying or enumerating certain microorganisms inside a sample containing selective microbial growth agents, chosen among salts (e.g. bile salts, sodium desoxycholate), other substances such as resins, natural plant extracts, fatty acids, esters, bactericides, bacteriostatics, alcohols, substances with superficial activity or their mixtures in quantities ranging from 0.0033 to 0.8 mg/g of three-dimensional structures of clays or ceramics and antibiotics (e.g. vancomycin, nadilixic acid), antifungals (e.g. nystatin, ketoconazole, amphotericin B), in quantities ranging from 0.033 to 0.33 μg/g.

Those devices used by passing aqueous samples through their three-dimensional structure, and which have components that are highly soluble in water, may contain substances that contribute to fixating the nutritional compound and the enzymatic markers to three-dimensional structures, such as alginates (e.g. sodium or calcium), natural polysaccharides; pectin, chitin, gum Arabic and other gums, starches such as pregelatinized corn starch, dextran and carboxymethylcellulose and other polymer derivatives of them; carrageenan, agar, agarose and artificial polymer derivatives, derivatives of vinyl alcohol, polybutylene, polyethylene and polypropylene, polyvinylpyrrolidone in quantities ranging from 0.01 to 0.5 gig of three-dimensional structure.

Other compounds may be part of the devices, such as those substances that increase their absorption capacity, like activated carbon and cellulose in the amount of 2 to 4 mg/g, which is used for forming devices whose structures have specific surfaces of less than 3×10 m²/m³.

A device may be formed by a three-dimensional structure or by a set of structures. Each three-dimensional structure may form a film or layer with a thickness of 5 nm to 1 mm; or a column up to 10 cm high; or particles with different geometric shapes, such as spheres, pearls, hexagons, cubes, with a diameter of 5 nm to 10 mm; cylinders or tubes with a diameter of 5 nm to 10 cm and a height of 5 nm to 10 cm; fibres, networks, or adopting the shape of the container that holds them. In some cases the structure of the device may increase its size and volume because it “swells” when it absorbs the sample containing the microorganisms, or the second solvent containing the microorganisms and occupying the entire volume of the container that holds it. These may be devices prepared from hydroxyapatite, agar and pregelatinized corn starch.

A device may have a three-dimensional structure that shows different zones with different porosities and different diameters or clearances of nano-, micro- and macrocavities throughout its volume, its length or its diameter, whereas said zones distribute as gradients or in differentiated zones.

The unique three-dimensional structure of each device contains one or more nutritional compounds and enzymatic markers different throughout its volume, length or diameter, whereas those compounds are distributed as gradients or in differentiated zones.

Devices may maintain their three-dimensional structure over supports shaped as sheets, layers or cylinders that may be pervious or impervious to them, or surrounded by impervious materials on at least 90% of their surface.

Some implementation examples are given below

### EXAMPLE 1

A sample of nutritional bases was taken for the bacterial growth promotion assay (E. coli), such as papain-hydrolyzed heart tissue, according to Cuban Invention Copyright Certificate No. 22442 in the amount of 0.2 g/L of deionised water, extracts of Saccharomyces cerevisiae yeast, according to Cuban Invention Copyright Certificate No. 22221, in the amount of 0.2 g/L, hydrolyzed enzymes of casein (Cuban Invention Copyright Certificates No. 22166) in the amount of 0.2 g/L and 0.2 g/L of pancreatic hydrolysate of heart.

Likewise, a mixture of all of them was formed in the following amount: papain-hydrolyzed heart tissue in the amount of 1 g/L of deionised water, extract of Saccharomyces cerevisiae yeast in the amount of 1 g/L, hydrolyzed enzymes of casein in the amount of 2 g/L and 1 g/L of pancreatic hydrolysate of heart.

Products to be tested were inoculated with 0.1 ml of a suspension of target microorganisms with a concentration of 3×10⁸ UFC/ml.

Bases and the mixture were incubated separately for 8 hours at 37° C. under an aerobic atmosphere, in which the increase of the optical density was monitored with a spectrophotometer at 680 nm.

Of all variants, the nutritional mixture showed a reduction of the E. coli lag growth phase in 30 minutes, while individual bases showed a variable duration on that phase, among them: papain-hydrolyzed heart tissue—45 minutes, extracts of Saccharomyces cerevisiae yeast—80 minutes, hydrolyzed enzymes of casein—60 minutes and pancreatic hydrolysate of heart—50 minutes.

Therefore, the mixture of nutritional components was selected, which hereinafter will be identified as CCL, and was dissolved in 1 L of deionised water as first solvent in the amount of 5 g/L (variant 1) and in the amount of 10 g/L (variant 2).

This nutritional mixture had already been added the pancreatic hydrolysate heart tissue in the amount of 1 g/L (variant 1) and 2 g/L (variant 2), resulting in the already mentioned nutrient concentrations of 5 g/L and 10 g/L, respectively.

Once the compound was prepared, two enzymatic markers were added, a chromogenic one [2-nitrophenyl-β-D-galactopyranoside (C₁₂H₁₅NO₆)], in quantities ranging from 0.5 g/L (variant 1) and 1 g/L (variant 2) and another fluorogenic [4-methylumbelliferyl-β-D-glucuronide (C₁₆H₁₆O₉.2H₂O)] in quantities ranging from 0.075 g/L (variant 1) and 0.15 g/L (variant 2).

Other substances were added to the mixtures of nutritional compounds and enzymatic markers, such as growth promoters, specifically lactose (5 and 10 g/L), sorbitol (0.5 and 1 g/L), L-tryptophan (1 and 2 g/1); inorganic salts, specifically monobasic potassium phosphate (2.75 and 5.5 g/l), dibasic potassium phosphate (2.75 and 5.5 g/L) and sodium chloride (5 and 10 g/L); finally, bile salts were added with a concentration of 1.3 to 2.6 g/L.

The nutritional compound selected, along with the enzymatic markers and other components, were dissolved in the first solvent in quantities ranging from 23.9 g/L for variant 1 and 47.8 g/L for variant 2.

Once the nutritional mixture is formed and the enzymatic markers and other components have been dissolved in the first solvent, they were sterilized by filtration.

Nutritional mixtures along with the enzymatic markers and other components dissolved in the first solvent were put in contact with two three-dimensional structures of ceramics, specifically hydroxyapatite that had been previously sterilized at 180° C. for 60 minutes.

Contact time between the compounds of variant 1 (V1) and of variant 2 (V2) was of 60 minutes.

These structures had a specific surface of 7500 m²/m³.

The dimensions of the three-dimensional structures mentioned above had combinations of all diameters or clearances, corresponding to nano- and microcavities with diameters or clearances ranging from 5 nm to 600 μm in the form of pores. These structures showed cylinder shapes with a 0.5 cm diameter and height of 0.5 cm.

When the absorption stage was completed, the first solvent was eliminated by drying the three-dimensional structures at a temperature of 60° C. in a vacuum oven for 3 hours.

The capacity of recovering the target microorganism (E. coli) was verified, proving that the structures had detection limits of less than 1 UFC/100 ml for both variants.

An E. coli suspension in saline isotonic solution with a concentration of 3×10⁶ UFC/ml was put into contact with 0.1 g of three-dimensional structures in quantities of 0.2 ml (2 ml/g ratio).

Afterwards, the three-dimensional structures were kept under a temperature of 35±2° C., under aerobic conditions throughout a 2-hour period that coincides with the duration of the lag growth phase of E. coli.

When the 2-hour incubation period was completed, the presence of the target microorganism was visually identified on both variants by its fluorescence under UV light at 366 nm over the supernatant liquid, where E. coli could be identified because of its positive reaction to glucuronidase (fluorescence).

### EXAMPLE 2

Similar to variant 1 of Example 1, although with the following differences:

V3—Hydroxyapatite pearls, with a total weight of 0.2 g and a specific surface of 2×10³ m²/m³, impregnated with the nutritional compound according to V2 of Example 1.

V4—Hydroxyapatite pearls, with a total weight of 0.2 g, with specific surface of 3000 m²/m³, impregnated with the nutritional compound according to V2 of Example 1.

The structures were left absorbing the nutritional compounds for 2 hours and were vacuum-dried for 2 hours at 60° C.

Afterwards, they were inoculated with 2 UFC/ml of E. coli in a volume of 0.2 ml (1 ml/g). The fluorescence of E. coli was observed after 120 minutes.

### EXAMPLE 3

V5—Hydroxyapatite pearls, with a total weight of 0.2 g and a specific surface impregnated with the nutritional compound according to V2 of Example 1.

V6—Hydroxyapatite pearls, with a total weight of 0.2 g and a specific surface of 1.5×10³ m²/m³, impregnated with the nutritional compound according to V2 of Example 1.

V7—Cellulose discs with no clays, with a 6-mm diameter, 0.94 cm² surface and 0.014 g, impregnated with the nutritional compound according to V1 of Example 1.

V8—Nutritional compound according to V1 of Example 1 in a 0.25 ml volume.

Ceramics were impregnated with the nutritional compound for 3 hours and the first solvent was eliminated at a temperature of 70° C.

Afterwards, they were inoculated with 0.1 ml of concentrated suspension (1 colony in 5 ml of saline solution) of E. coli.

As a result, fluorescence was observed after 90 minutes on V6, after 105 minutes on V5 and it was not observed either on V7 or on V8, which proves that the combination of using three-dimensional clays along with the selected nutritional mixtures that reduce the lag growth phase accelerate microbial detection and identification in comparison with using only the compound, or of this with any other kind of structure.

### EXAMPLE 4

In general, this method was carried out according to Example 1, with the following differences:

The bacterial growth promotion assay was carried out with papain-hydrolyzed heart tissue, according to Cuban Invention Copyright Certificate No. 22442 in the amount of 0.2 g/L of deionised water.

This base was incubated for 8 hours at 37° C. under an aerobic atmosphere and the increase of optical density was monitored with a spectrophotometer at 680 nm.

The nutritional base showed a reduction of the lag growth phase of Enterococcus after 120 minutes.

This hydrolyzed was chosen for the preparation of the device (variant 9) in order to carry out this method, and it was dissolved in 1 L of deionised water as first solvent in the amount of (10 g/L equivalent to 10 mg/g of structure) and salts were added in order to regulate a potential pH change caused by the three-dimensional structure, specifically dipotassium phosphate (3.5 g/L, equivalent to 8.75 mg/g of structure), potassium phosphate (1.5 g/L, equivalent to 3.75 mg/g of structure) and sodium chloride (5 g/L, equivalent to 12.5 mg/g of structure), which makes for a total nutritional mixture quantity of 50 mg/g). To the structure was added methylumbelliferyl-β-glucoside in the amount of 0.075 g/L as fluorogenic marker, equivalent to 0.1875 mg/g of three-dimensional clay structure.

All components were previously sterilized in an autoclave at 121° C. for 15 minutes.

The presence of Enterococcus was observed because of the bluish fluorescence that appeared after 120 minutes.

### EXAMPLE 5

In general, the method was carried out according to Example 4, with the following differences:

In order to test the bacterial growth promotion (Enterococcus faecalis ATCC 29212, Enterococcus faecium ATCC 19434, Enterococcus avium ATCC 14025), papain-hydrolyzed heart tissue was used according to Cuban Invention Copyright Certificate No. 22442, in the amount of 0.2 g/L of deionised water.

The bases were incubated for 8 hours at 37° C. under an aerobic atmosphere and the increase of optical density was monitored with a spectrophotometer at 680 nm.

The nutritional bases showed a reduction of the lag growth phase of 2 hours. The devices were prepared for carrying out the method, duplicating the concentrations of each component that was embedded in the structures.

After 90 minutes, a change of color was observed in the second solvent, which turned slightly bluish in regards to the original that was greenish on both devices with three-dimensional clay structures for E. avium.

After 150 minutes, the appearance of fluorescence was observed in the second solvent for the device with 7.5×10³ m²/m³ specific surface (variant 10) and slight fluorescence for the device with specific surface of 2×10³ m²/m³ (variant 11) for E. avium.

After 210 minutes, E. faecalis was detected in the device of variant 11 due to the appearance of fluorescence.

After 240 minutes, all microorganisms showed fluorescence on their three-dimensional structures.

### EXAMPLE 6

Carried out according to Example 1, with the following differences:

Three devices were prepared: the one described in variant 11 (V11) with a specific surface of 2×10³ m²/m³, the one described in variant 12 (V12) with a specific surface of 3.3×10³ m²/m³ and the one described in variant 13 with a specific surface of 1.5×10³ m²/m³.

Contact time of the compounds of these variants was 180 minutes.

When the absorption was concluded, the first solvent was eliminated by drying the three-dimensional structures at a temperature of 60° C. in a vacuum oven over a 60-minute period.

An E. coli suspension on a saline isotonic solution was put in contact with a concentration of 1 colony on 5 ml, from which 0.1 ml was taken and applied over the surface of the device.

After 90 minutes, fluorescence was observed on the device with the structure of variant 11 (V11).

After 210 minutes, fluorescence was observed on the other two devices (V12 and V13) with structures of specific surface of 1.5×10³ m²/m³ to 3.3×10³ m²/m³.

### EXAMPLE 7

Similar to Variant 1 of Example 1, with the difference that the following variants were formed:

V14—Kaolinite compacted in agglomerates, with a total weight of 0.2 g, impregnated with the nutritional compound according to V2 of Example 1.

Spaces between kaolinite particles form cavities of different shapes, with sizes corresponding to micro- and macrocavities.

V15—Powdered bentonite, ground on a ball mill and later compacted with a total weight of 0.2 g, impregnated with the nutritional compound according to V2 of Example 1. Spaces between kaolinite particles form cavities of different shapes, with sizes corresponding to macrocavities.

Structures are left absorbing the nutritional compounds over 4 hours and vacuum-dried for 3 hours at 60° C.

An E. coli suspension is put in contact with an isotonic sodium phosphate solution with a concentration of 10⁶ UFC/ml with 0.1 g of three-dimensional structures in quantities ranging from 0.2 ml (relation of 2 ml/g).

E. coli fluorescence is observed at 280 minutes.

### EXAMPLE 8

A strain of E. coli ATCC 25922 is tested as described in Example 1, and which is formed according to V2 of that example, along with a nutritional compound prepared as described in said variant 2 of Example 1, with the difference that only fluorogenic substrate 4-methylumbelliferyl-β-D-glucuronide (MUG) is added in the amount of 0.2 g/L.

The nutritional mixture, along with the enzymatic marker and other components dissolved in the first solvent are put into contact with 3 three-dimensional structures.

The first structure of artificial ceramic nature is formed by a nano-layer of hydroxyapatite with nanoporosity, a specific surface of 50×10⁶ m²/m³ and 20 nm high nanocavities, resting over a lower agaropectin layer in the amount of 0.5 g/g. The structure also rests over the entire surface of a 6-cm diameter disc that is impervious to cellulose derivatives, specifically cellulose nitrate, with which an initial device is formed (variant 16).

The second structure is made of siliceous earth, with a specific surface of 3×10⁵ m²/m³ to 1×10⁶ m²/m³, and nano- and microcavity porosity, on which the nutritional compound is mixed with agar (0.3 g/g) dissolved in the first solvent. The second device is prepared by placing the siliceous earth structure described in a cylinder 3-mm high with a diameter of 90 mm (variant 17).

The third three-dimensional structure is formed by hydroxyapatite with a minimum specific surface of 1.25×10³ m²/m³ with irregular macro- and microcavities, followed by a three-dimensional zeolite structure with a specific surface larger than 5×10³ m²/m³. The nutritional compounds of Example 1, variant 2 and the fluorogenic substrate described in said variant are absorbed on the first and second structures. The overall height of these three-dimensional structures reaches 10 cm with a 4-cm diameter, forming a device in which this three-dimensional column is placed in a tube made of impervious material with openings on its upper and lower portions, in such a way that the tube of impervious material (PVC) surrounds 90% of the external surface of the structure (variant 18).

An E. coli suspension is put into contact with a saline isotonic solution with a concentration of 10² UFC/ml over the entire surface of the first device (V16), aided with a swab soaked in sodium alginate. The three-dimensional structure is then maintained under a temperature of 35±2° C. and aerobic conditions over a 120-minute period, whereas growth is detected by the emission of fluorescence under light at 366 nm aided by a sensor and identified by glucuronidase activity.

An E. coli suspension is put into contact with a saline isotonic solution with a concentration of 10 UFC/ml over the entire surface of the second device (V17), aided by an automatic pipette and is distributed with a Drigalski spatula. The three-dimensional structure is then maintained at temperatures of 35±2° C., under aerobic conditions over a 240-minute period, whereas growth is detected by the emission of fluorescence under light at 366 nm, aided by a sensor and identified by glucuronidase activity.

A 10-L sample of deionised water, artificially contaminated with E. coli at a concentration of 8 UFC/10 L, is passed throughout the entire volume of the device and is left for incubation under the conditions described in the previous experiments. After 210 minutes, fluorescence was observed inside the device.

### EXAMPLE 9

Four three-dimensional structures of natural clays or ceramics were evaluated with two nutritional compounds for identifying Gram-negative bacteria.

Nutritional components were selected according to Example 1.

Different devices were prepared with this compound in order to implement the method.

The selected supports were HAP-S(specific surface of 7.5×10³ m²/m³) and HAP-56 (specific surface of 3×10⁵ m²/m³) ceramics, as well as siliceous earth purified and calcined (TSC) (specific surface of 5.3×10⁴ m²/m³).

A nutritional mixture was prepared with fluorogenic and chromogenic markers and other components according to V1 of Example 1. In parallel, another mixture of a fluorogenic compound with salts and other components according to the following compound was prepared: ammonium sulfate [(NH₄)₂SO₄] 5.0 g/L of the first solvent; hydrogen potassium phosphate [K₂HPO₄] 0.45 g/L; dihydrogen potassium phosphate [KH₂PO₄] 0.31 g/L; hydrogen sodium phosphate [Na₂HPO₄] 0.92 g/L; sodium chloride [NaCl] 0.1 g/L; calcium chloride [CaCl₂] 0.05 g/L; heptahydrated magnesium sulfate [MgSO₄.7H₂O] 0.2 g/L; L-histidine monohydrochloride 0.005 g/L; L-tryptophan 0.02 g/L; L-methionine 0.02 g/L, dextrose 10.0 g/L and MUG in the amount of 0.2 g/L.

The pH of both compounds was adjusted to 6.8, and they were sterilized by filtration through Nalgene disposable filtration units (0.2 μm, pore size) (Nalge Co., Rochester, N.Y.).

The three-dimensional structures were prepared with the mixtures using the following methodology:
1 g of each structured support was weighed in dry-heat resistant containers.
They were sterilized at 180° C. for 1 hour, then 2 ml of the nutritional compound or the enzymatic marker mixture with salts and other components (MCS) sterilized by filtration under vertical laminar flow were added and were left to embed in the structure for 1 hour; the lid was removed and they were left under vertical laminar flow for 3 hours until the first solvent was eliminated. A 0.1 g portion of the device was then stored under aseptic conditions, in such a way that its height and diameter reached 0.5 cm, thus obtaining a glass container with hydrolytic quality 1, transparent, with a lid and maximum capacity of 2 ml.

The final devices contained: ammonium sulfate [(NH₄)₂SO₄] 10 mg/g of three-dimensional structure; hydrogen potassium phosphate [K₂HPO₄] 0.9 mg/g; dihydrogen potassium phosphate [KH₂PO_{4] 0.62} mg/g; hydrogen sodium phosphate [Na₂HPO₄] 1.84 mg/g; sodium chloride [NaCl] 0.2 mg/g; calcium chloride [CaCl₂] 0.1 mg/g; heptahydrated magnesium sulfate [MgSO₄.7H₂O] 0.4 mg/g; L-histidine monohydrochloride 0.001 mg/g; L-tryptophan 0.04 mg/g; L-methionine 0.04 mg/g and dextrose 20.0 mg/g and MUG in the amount of 0.4 mg/g, for un total of 34.55 mg/g.

The design of the experiment covered the following assay variants:

| Types of support | Nutritional compound | MCS |
|---|---|---|
| HAP-S | V18 | V22 |
| HAP-56 | V19 | V23 |
| TSC | V20 | V24 |

In order to inoculate all variants of the study, microbial suspensions of approximately 10⁸ UFC/ml were prepared in 9 ml of a sterile dual salt 0.85% (pip) solution from pure cultures of Escherichia coli ATCC 25922 incubated for up to 24 hours. The volume of the inoculum was 0.2 ml for each assay variant, ensuring an inoculum concentration of 2×10⁷. Inoculated devices were incubated at 35±2° C. under aerobic conditions.

Readings were made visually, using a 366 nm UV lamp every 30 minutes.

The results obtained are shown on the following table, stating the period in hours and minutes of positive response to fluorescence.

| Devices with mixture + | | | | | | |
|---|---|---|---|---|---|---|
| markers + other components | | | | MCS Devices | | |
| HAP-S | HAP-56 | TSC | | HAP-S | HAP-56 | TSC |
| (V18) | (V19) | (V20) | Microorganism | (V22) | (V23) | (V24) |
| 2:30 | 3:30 | 2:30 | E. coli | 24:00 | 5:00 | 24:00 |

This indicator shows that the microbial species has enzymatic activity that corresponds to the enzymatic marker used on the compounds.

It is also proven that it is not obvious that each nutritional compound combined with a three-dimensional clay structure responds in a shorter time or detects or identifies microorganisms in a shorter period.

It is also proven that the combination of clay or ceramic structures with nutritional mixtures, enzymatic markers and other ingredients accelerates identification in regards to V21 to V23 variants that did not contain nutritional components based on protein hydrolyzed and extracts that were previously protected by the authors of this invention, thus proving that it is paramount to use compounds that allow shortening the lag growth phase of bacteria by reducing the detection and identification time.

This experiment also proved that, surprisingly, the nutritional compounds designed are capable of attenuating or eliminating the inhibitor effect or antibacterial application of this structure, as described by different authors for certain biotechnological processes, in particular that of zeolite (V21) when a nutritional compound especially designed for eliminating this antibacterial effect is added.

### EXAMPLE 10

The four three-dimensional structures of natural clays or ceramics were evaluated with two nutritional compounds for identifying the Gram-negative bacteria described in the previous example (Example 9).

The selection of nutritional components was likewise carried out in accordance with the methodology of Example 1, but with a strain of Pseudomonas aeruginosa (ATCC 27853). Individual and mixed hydrolyzed enzymes were evaluated, among them papain-hydrolyzed of beef heart tissue, according to Cuban Invention Copyright Certificate No. 22442, and pancreatic hydrolysate heart tissue and their mixtures. Results showed that for these 3 cases the lag phase had a maximum duration of 120 minutes; therefore, both were selected for preparing the nutritional compound.

Different devices were prepared with this compound in order to implement the method.

A nutritional mixture was prepared with fluorogenic and chromogenic markers and other components according to V1 of Example 1. In parallel, another mixture of a fluorogenic compound was prepared with salts and other components according to the description of the previous example (MCS).

All other devices and inocula were prepared according to the descriptions of Example 9, with the difference that the inoculum in the devices of Pseudomonas aeruginosa ATCC 27853 and of Enterococcus faecalis ATCC 29212 were added to the experiment.

The design of the experiment covered the following assay variants:

| Types of support | Nutritional compound | MCS |
|---|---|---|
| HAP-S | V18 | V21 |
| HAP-56 | V19 | V22 |
| TSC | V20 | V23 |

The results obtained are showed on the following table, stating the period in hours and minutes of positive response to fluorescence.

| Devices with mixture + | | | | | | |
|---|---|---|---|---|---|---|
| markers + other components | | | | MCS Devices | | |
| HAP-S | HAP-56 | TSC | | HAP-S | HAP-56 | TSC |
| (V18) | (V19) | (V20) | Microorganism | (V22) | (V23) | (V24) |
| 3:00 | 24:00 | − | P. aeruginosa | − | 3:00 | − |
| − | − | − | E. faecalis | − | − | − |

The unexpected appearance of greenish fluorescence of Pseudomonas was observed in the three-dimensional ceramic structures after only 3 hours in variant V18; this effect had never been achieved with any other known diagnostic tool.

Secondly, it was proven that in order to detect and identify certain microorganisms in as little time as possible, the specific surface and size of cavities (3 hours for V18 and 24 hours for V19) is of the essence, as well as the dependence on this specific type of clay combined with a mixture for eliminating the inhibitor effect of these clays or ceramics (detected in V18 and not detected in V20 or V21).

It was also proven that the combination of two structures with different compounds in the same device (V19+V22) finally lead to identifying the target microorganisms.

It was proven once more that specific nutritional compounds selected among those that shorten the lag growth phase to a few minutes ensure detection, identification and recuperation in a few minutes (280 minutes).

The selective character of this device was proven, as it inhibited E. faecalis, because the compound embedded in the clay structure, along with it, were able to inhibit it—even with concentrations as high as 10⁷ UFC/ml.

### EXAMPLE 11

Evaluation of three kinds of three-dimensional structures of natural clays or ceramics (HAP-S, HAP-56 and TSC) with two nutritional compounds for the identification of Gram-positive bacteria.

Different mixtures of nutritional bases were used for testing bacterial growth promotion (Staphylococcus aureus and Streptococcus pyogenes), among them extract of Saccharomyces cerevisiae obtained through enzyme hydrolysis as described in Cuban Invention Copyright Certificate No. 22221, hydrolyzed enzymes of bovine blood (Cuban Invention Copyright Certificate No. 22208), hydrolyzed enzymes of casein (Cuban Invention Copyright Certificates No. 22089, hydrolyzed enzymes of commercial soy and commercial meat extract.

The first mixture (M1) contained: extract of Saccharomyces cerevisiae—3.24 g/L; hydrolyzed enzymes of bovine blood—6.37 g/L; hydrolyzed enzymes of casein—9.97 g/L; commercial soy peptone—3.24 g/L and commercial meat extract—2.43 g/L.

The second mixture (M2) contained: extracts of Saccharomyces cerevisiae—5.0 g/L; hydrolyzed enzymes of bovine blood—5.0 g/L; hydrolyzed enzymes of casein—5.0 g/L; commercial soy peptone—6.0 g/L and commercial meat extract—3.0 g/L.

The third mixture (M3) contained: extracts of Saccharomyces cerevisiae—6.0 g/L; hydrolyzed enzymes of bovine blood—6.0 g/L; hydrolyzed enzymes of casein—3.0 g/L; commercial soy peptone—6.0 g/L and commercial meat extract—4.0 g/L.

The products to be tested were inoculated with 0.1 ml of a suspension of target microorganisms with a concentration of 3×10⁸ UFC/ml.

The mixture and the bases were incubated separately for 8 hours at 37° C. under an aerobic atmosphere, and the increase of optical density was monitored with a spectrophotometer at 680 nm.

All variants shortened the lag phase to only 1 hour for S. pyogenes, and the duration of this phase for S. aureus was only 2 hours. M3 was chosen among these, since the growth of S. pyogenes was slightly more intense in it.

The following nutritional compounds were tested:
Nutritional compound (STR-STAP) formed by: hydrolyzed enzymes of casein, according to Cuban Invention Copyright Certificate No. 22166 (3.0 g/1); extract of Saccharomyces cerevisiae yeast, according to Cuban Invention Copyright Certificate No. 22221 (6.0 g/l); and hydrolyzed blood enzymes, according to Cuban Invention Copyright Certificate No. 22208 (6.0 g/1). In addition, commercial meat extract (4.0 g/l); papain-digested soy proteins (6.0 g/1) were also added, along with other components, such as thallium acetate (0.014 g/1); nalidixic acid sodium salt (0.008 g/L); DL-phenylalanine (1.0 g/L); ammonium ferric citrate (0.5 g/l); sodium chloride (0.2 g/), dextrose (0.1 g/L) and 0.2 g/L of MU-phosphate were added as fluorogenic marker;
Additional synthetic compound with no hydrolyzed or extracts (MCS according to the previous example) with the addition of 4-methylumbelliferyl phosphate (MU-phosphate) in the amount of 0.2 g.

Compounds were dissolved in direct proportion with a first solvent (deionised water) whose pH was adjusted to 7.3. They were sterilized by filtration using 0.2 μm disposable filtration units. The devices were prepared following the methodology described in Example 10, and the design of the experiment covered the following assay variants:

| | Compound | |
|---|---|---|
| | MU-phosphate | MU-phosphate |
| Structure | STR-STAP | MSC |
| HAP-S | V26 | V29 |
| TSC | V27 | V30 |
| No three-dimensional | V28 | |
| structure | | |

Target species Staphylococcus aureus ATCC 25923 and Streptococcus pyogenes ATCC 19615 were chosen for this experiment. The preparation of their dilutions, inoculation and incubation were conducted as described in Example 10.

The following tables show the fluorescence development period (in hh:mm) for each of the microbial species evaluated against each enzymatic marker, using only those compounds from variants 24 to 27—this is, only those containing the nutritional mixture.

| | | No three- | |
|---|---|---|---|
| | Device | dimensional | |
| HAP-S | TSC | structure | Microorganism |
| 3:00 | 5:00 | 24:00 | S. aureus |
| 4:00* | 24:00* | — | S. pyogenes |

*weak but noticeable fluorescence

The evaluation carried out with this fluorogenic substrate proved that the most satisfactory variants—in terms of response speed—were the combinations of STR-STAP with HAP-S (variant 24) that in only 3 to 5 hours of incubation allowed detection of S. aureus and S. pyogenes.

Variant 25 allowed detecting microorganisms with very high nutritional requirements, such as S. aureus in only 5 hours and S. pyogenes in only 24 hours.

Once more it was proven that only the combination of nutritional mixtures with enzymatic markers and three-dimensional ceramic structures (V24-V25) are capable of accelerating microbial detection when compared to compounds alone (V26), or even achieving their detection (V26 negative for S. pyogenes).

Other combinations were prepared for creating compound devices with two structures and two different compounds: one containing hydrolyzed and extracts, and the other synthetic:

### V24+V27=V29

### V25+V28=V30

### V24+V28=V31

### V25+V27=V32

The combination of structures from the studied variants brought better results in terms of detection time for one of the combined devices, so it was not necessary to combine all the others, as described below:
the combination of HAP-s with the original nutritional compound and the same three-dimensional structure, although with the synthetic compound (V32), reduced the detection time of S. aureus from 5 hours to 3 hours, and the one for S. pyogenes from 24 hours to 4.30 hours.

### EXAMPLE 12

Study of the response from different devices and the method before a urine sample. The devices were formed using four different kinds of three-dimensional structures of natural and artificial clays and ceramics (HAP-S, HAP-56) each combined with the compound described in variant 1 of Example 1 and carrying out the method as described in Example 9.

The inoculum used was 0.2 ml of the sample for each variant.

In parallel, the sample was evaluated using the traditional procedure, using agarized media such as CromoCen CC and bromothymol blue lactose agar (ABL).

In both testing schemes (the new method with the clay device and the traditional method), the inoculated samples were incubated at 35° C. Test readings were taken every 30 minutes after applying the device, using a 366 nm UV lamp.

The following table shows the period (in hh:mm) after which fluorescence was detected, which proved the presence of infection in the clinical sample, related with a positive species on the enzymatic marker used.

| HAP-S + CCL (V33) | HAP-56 + CCL (V34) |
|---|---|
| 3:00 | 2:30 |

The combination of CCL with HAP-56 was the variant that responded faster, in 2 hours and 30 minutes, followed by the combination with the HAP-S structure that was 3 hours. This result indicates that E. coli is the bacteria that caused the infection, considering the selectivity of the nutritional compound and of the device, and that in most cases on record this kind of sample responds to this species.

E. coli was only detected in both media (CromoCen CC and ABL) after 24 hours when using conventional procedures.

This proves how accurate the method and the device are, and that it accelerated the procedure by at least 8 times.

### EXAMPLE 13

Study of the relationship between the nutritional capacity of the compounds and the development of microbial enzymatic activity over an enzymatic marker.

For this purpose, the nano-compound CCL with HAP-S, prepared as described in Example 9, was selected. On the other hand, a variant using HAP-S as support was prepared, in which an aqueous solution of MUG with a concentration of 02 g/L (p/v) was added and then was dehydrated following the same methodology described in Example 9.

A pure culture of E. coli was used as assay microorganism, from which a 10⁸ UFC/ml suspension was prepared. 0.2 and 0.4 ml volumes were used for the study, producing inocula that were applied on the structures of up to 10⁷, and then both devices were inoculated in parallel: CCL with HAP-S(V35) and MUG with HAP-S(V36).

The variants were incubated at 35° C., observing their response to fluorescence every 30 minutes, using a 366 nm UV lamp.

The results obtained are shown in the following table, highlighting the time in hh:mm after which a positive response to fluorescence was observed.

| Inoculum volume (ml)/final density | 0.2 ml/10⁷ | 0.4 ml/10⁸ |
|---|---|---|
| CCL/HAP-S | 2:00 | 1:30 |
| MUG/HAP-S | 4:00 | 3:30 |

This experiment proved the significant influence of the combination of hydrolyzed or extracts obtained from nutritional substances of protein nature present on the nutritional compound used. This allowed the microbial species to adapt faster to the conditions of the device and showed enzymatic activity during its development within the lag phase (2 h) that allowed in this case its identification before the enzymatic marker used for a minimum period of 1 hour and 30 minutes. Nevertheless, for the structure that contained only the fluorogenic substrate, its response was detected 2 hours later, since this is not based on the activation of enzymatic mechanisms, but on detecting them at much higher concentrations, which is the method used in the state-of-the-art solutions mentioned above.

### EXAMPLE 14

Study of the influence of pH of the compound over the action of the fluorogenic substrate for revealing microbial enzymatic activity.

Using the compound CCL described in Example 9, four experimental variants were prepared with different pH values (6.6-6.8-7.0-7.2). Each variant was sterilized through filtration and was independently embedded in the HAP-S ceramic (variant 37) and inoculated and incubated following the procedure mentioned in Example 9.

The results obtained are showed in the following table, stating the period (hh:mm) of positive response to fluorescence as indicator of microbial enzymatic activity over the marker used,

| Compound pH | 6.6 | 6.8 | 7.0 | 7.2 |
|---|---|---|---|---|
| CCL/HAP-S | 1:30 | 1:30 | 2:00 | 1:30 |

The results show that there is no significant influence of the compound pH on the detection of the enzymatic activity of the (E. coli) microorganism with the enzymatic marker used (MUG).

Positive response was detected in the period between 1 hour and 30 minutes to 2 hours of culture.

### EXAMPLE 15

Evaluation of four three-dimensional structures of natural clays or ceramics (HAP-S, HAP-56 and TSC) using two nutritional compounds and two enzymatic markers for independent tests aimed to identify a species of the Candida genre.

In order to select the nutritional compound(s), the reduction of the lag phase duration with vegetal extracts of sweet potatoes that was previously developed by the authors of this invention, namely hydrolyzed enzyme of casein, soy peptone, a mixture of yeast peptones and hydrolyzed enzymes, all in quantities ranging from 0.2 g/L was studied. Optical density was monitored every 1 hour with a 380 nm spectrophotometer. The results showed that the extracts of sweet potatoes shortened the lag phase for C. albicans by at least 1 hour from all other comp components, requiring only 16 hours.

The first experimental variants were prepared using the fluorogenic MU-phosphate substrate as the first enzymatic marker. The substrate was added to the CND compound in the amount of 0.2 g, formed by: extract of sweet potatoes 20.0 g; extract of Saccharomyces cerevisiae yeast 10.0 g; potassium phosphate 1.0 g; magnesium sulfate 0.5 g; sodium desoxycholate 0.5 g and nalidixic acid 0.03 g, for one liter of deionised water.

The other compound used was the MCS medium, to which was added the same quantity of MU-phosphate (0.2 g/1).

Similar variants were prepared in parallel, using L-proline methylcoumarin (L-Pro) as enzymatic marker substrate, 0.2 g of which were added to the CND and MCS compounds, respectively.

The nutritional compounds prepared with the enzymatic markers and other ingredients prepared for each experimental variant were proportionally dissolved with one liter of deionised water as first solvent and their pH was adjusted to 6.6. They were sterilized by filtration using 0.2 μm disposable filtration units.

Structures were prepared following the methodology described in Example 9 and the design of the experiment covered the following assay variants:

| | Enzymatic marker and culture medium | | | |
|---|---|---|---|---|
| | MU-phosphate | | L-proline | |
| Structure | CND | MCS | CND | MCS |
| HAP-S | V38 | V41 | V44 | V47 |
| HAP-56 | V39 | V42 | V45 | V48 |
| TSC | V40 | V43 | V46 | V49 |

The microbiological evaluation was conducted with the reference strains: Candida albicans ATCC 10231, Candida parapsilosis ATCC 22019 and Candida glabrata ATCC 15126, just harvested from Sabouraud dextrose agar for 36 hours. Suspensions from these cultures were prepared in 9 ml tubes with a sterile dual salt 0.85% (p/p) solution until a microbial density of 10⁸ UFC/ml was achieved.

A 0.2 ml volume was inoculated with each variant of the devices according to the method and incubated at 35° C. Fluorescent response was recorded every 30 minutes using a 366 nm UV lamp.

The results obtained are shown independently for each enzymatic marker in the following tables, showing the period (hh:mm) in which a positive response to the fluorescent reaction was observed.

Fluorogenic substrate: MU-phosphate

| CND medium | | | | MCS medium | | |
|---|---|---|---|---|---|---|
| HAP-S | HAP-56 | TSC | | HAP-S | HAP-56 | TSC |
| (V38) | (V39) | (V40) | Microorganism | (V41) | (V42) | (V43) |
| − | NU | − | C. albicans | − | NU | − |
| 15:00* | NU | − | C. parapsilosis | 15:00* | NU | − |
| 15:00* | NU | − | C. glabrata | 15:00* | NU | − |

*perceptible fluorescence,
NU: not useful,
− negative response

First of all, it should be noted that the three Candida species evaluated exhibited phosphatase activity; therefore, in all cases variants were examined with the purpose of detecting positive response to fluorescence before the MU-phosphate substrate.

On the other hand, once more the evaluation of this fluorogenic substrate (MU-phosphate) proved its decay before the HAP-56 ceramic, because since the nano-compound was hydrated, it revealed the appearance of positive response to fluorescence, which is not related to the enzymatic action of the microbial species. This nullifies the reading from the outset, since it was reported as “not useful.”

On the other hand, TSC clay somehow blocks the response either from the enzymatic activity of the tested microorganisms or of the specific fluorogenic substrate, since the biological functionality of the nano-compound is not observed throughout the entire culture period.

Regarding HAP-S ceramic, a similar response was detected for each tested compound—although it was different for each Candida species.

With C. albicans it was detected that the species was not able to show its activity over the MU-phosphate substrate, while with the C. parapsilosis and C. glabrata species fluorescence appeared after 15 hours of culture—although with very low intensity, along with the appearance of an indicator that did not increase its intensity during a larger incubation period.

In general, the response obtained from using this enzymatic marker (MU-phosphate) as part of the nutritional compounds is closely related to the structure used as nano-structured support; therefore, the response from Z ends up being the most convenient one.

The following table shows the results found with fluorogenic substrate L-Pro.

Fluorogenic substrate: L-Pro

| CND medium | | | | MCS medium | | |
|---|---|---|---|---|---|---|
| HAP-S | HAP-56 | TSC | | HAP-S | HAP-56 | TSC |
| (V44) | (V45) | (V46) | Microorganism | (V47) | (V48) | (V49) |
| 15:00 | 15:00 | 15:00 | C. albicans | 15:00 | 15:00 | 15:00 |
| 15:00 | 15:00^{a} | 15:00 | C. parapsilosis | 15:00* | 15:00^{a} | 15:00 |
| − | − | − | C. glabrata | − | − | − |

*weak but perceptible fluorescence,
^{a}fluorescence with greater intensity

According to other studies done by the authors of this invention for detecting the enzymatic activity of several Candida species, it is well known that the C. albicans and C. parapsilosis species have enzymatic L-proline amidase. On the other hand, C. glabrata does not have the action of said enzyme.

Based on this prior knowledge, a reading was made, looking for a positive response to the fluorescence for the C. albicans and C. parapsilosis species.

It was noticed that the variants produced with the HAP-S, HAP-56 and TSC structures for both culture media (CND and MCS) showed satisfactory results by allowing the detection of C. albicans and C. parapsilosis in only 15 hours. Nevertheless, the greater intensity of the fluorescent response when using the HAP-56 ceramic is noticed in the original case.

Regarding the use of zeolite as support with this fluorogenic substrate, an incompatibility was found since it did not reflect microbial activity nor did it block in any way the decay of the L-Pro substrate, which makes the microbiological functionality of the nano-compound not ideal.

### EXAMPLE 16

Similar to variant 1 of Example 1, with the difference of the following variants:

V50—set of natural zeolite clays compressed as a tablet, with a total weight of 0.2 g and a specific surface of 3×10⁵ m²/m³ impregnated with the nutritional compound according to V1 of Example 1.

V51 powdered carbonateapatite [Ca₅(PO₄,CO₃)₃(OH)], placed on a 1 cm high and 1 cm tall pot, with a weight of 0.5 g and a specific surface of 4×10³ m²/m³ and 700 μm cavity size, impregnated with the nutritional compound according to V2 of Example 1.

V52—zeolite cubes with a total weight of 0.2 g and a specific surface of 7.0×10³ m²/m³, impregnated with the nutritional compound according to V44 of Example 15.

The structures were left absorbing the nutritional compounds for 1 hour and were vacuum dried for 3 hours at 60° C.

They were inoculated with 106 UFC/ml of E. coli inoculum (V50 and V51) and a 0.2 ml (1 ml/g) volume of C. albicans (V52).

E. coli fluorescence is observed after 180 minutes in V50 and 210 minutes in V51, and that of C. albicans is observed in V52 after 18 hours.

### EXAMPLE 17

Bacteria such as E. coli, E. coli 0157:117, Aeromonas hydrophila, Enterococcus avium and the filamentous fungi Aspergillus niger were taken for a bacterial growth test.

Each one of these microorganisms is tested with different nutritional bases, such as papain-hydrolyzed heart tissue, according to Cuban Invention Copyright Certificate No. 22442 in the amount of 0.2 g/L of deionised water; extracts of Saccharomyces cerevisiae yeast according to Cuban. Invention Copyright Certificate No. 22221, in the amount of 0.2 g/L; hydrolyzed enzymes of casein (Cuban Invention Copyright Certificates No. 22166); 0.2 g/L of sweet potato extract, as disclosed in Cuban patent No. 23507; tomato extract in the amount of 0.2 g/L (Cuban Invention Copyright Certificate No. 22308); and hydrolyzed enzymes of bovine blood (Cuban Invention Copyright Certificate No. 22208) in the amount of 0.2 g/L. Likewise, a mixture of all of them was formed in quantities ranging from 0.2 g/L of each one of them.

The bases and the mixture were incubated separately for 8 hours at 37° C. under an aerobic atmosphere, and the increase of optical density was monitored with a spectrophotometer at 680 nm.

Of all the variants, the nutritional mixture showed a reduction of the lag growth phase of all microorganisms after 90 minutes, with the exception of Aspergillus while the individual bases showed a variable phase duration, and in some cases higher than 2 hours; therefore, this mixture was chosen for the experiments.

This mixture is dissolved on a saline solution (NaCl with 9.5 g/L) in the amount of 10 g/L.

As soon as the nutritional compound was prepared, it was added different enzymatic markers, one chromogenic [2-nitrophenyl-β-D-galactopyranoside (C₁₂H₁₅NO₆)], in quantities ranging from 1 g/L and three fluorogenic (4-methylumbelliferil-β-D-glucoronide, 4-methylumbelliferyl-β-D-galactoside and 4-methylumbelliferyl-β-D-glucoside) in quantities ranging from 0.2 g/L each one.

Other substances were added to the mixtures of the nutritional compounds and enzymatic markers, such as growth promoters, specifically glucose (10 g/L); inorganic salts, specifically monobasic potassium phosphate (5.5 g/L) and dibasic potassium phosphate (5.5 g/L). The nutritional compound selected, along with the enzymatic markers and other components, are found dissolved in the first solvent in quantities ranging from 32.6 g/L.

Once the nutritional mixture is formed and along with the enzymatic markers and other components dissolved in the first solvent, they are sterilized by filtration. Nutritional mixtures along with the enzymatic markers and other components dissolved in the first solvent are put into contact with one and multiple three-dimensional structures of artificial ceramics, specifically the calcined hydroxyapatite that had been previously sterilized at 180° C. for 60 minutes.

Contact time of the compound is of 30 minutes.

This structure has a specific surface of 5×10³ m²/m³ and is formed by a variety of nano, micro- and macrocavities.

The three-dimensional structure has cavity dimensions that correspond to different combinations of all cavity diameters or clearances corresponding to nano-, semimicro- and microcavities with diameters or clearances of 5 nm to 10 μm with pore shapes. These structures have a cylinder shape, with a 100 cm diameter and height of 2 cm.

When the absorption was completed, the first solvent was eliminated by drying the three-dimensional structures at a temperature of 60° C. in a vacuum oven over a 3-hour period.

The capacity of recovering target microorganisms is verified after proving that the structures have detection limits of less than 1 UFC/100 ml by filtering 1 L of an artificially inoculated E, Coli suspension with a concentration of up to 6 UFC, which means that there are 0.6 UFC for every 100 milliliters, whereas they can be detected by fluorescence and through the yellowish hue of the structure.

For this assay, the suspensions of target microorganisms in saline isotonic solution with a concentration of 3×10⁶ UFC/ml are put into contact with the three-dimensional structures in quantities ranging from 1 ml and are distributed throughout the entire surface.

The three-dimensional structures are then maintained at temperatures of 35±2° C., under aerobic conditions for a period of up to 4 hours that coincides with the duration of the lag growth phase of E. coli.

When the incubation is completed for 4 hours at the most, the presence of target microorganisms is detected on all variants, on one case inoculated individually with each microorganism and on another case with the mixture of all of them. In the case of E. coli, fluorescence and color change of the structure to a yellowish hue were observed; for E. coli 0157:H7 and Aeromonas hydrophila, only the color change of the structure was observed. In all cases, detection took place after 2 hours.

In the case of Enterococcus avium, blue fluorescence with no color change was observed in the structure after 3 hours, and the filamentous fungi grown like a black structure over the surface of the device, although a yellowish hue was observed first on the growth zone. In the event that the sample is inoculated with the mixture of microorganisms, all reactions can be observed.

### EXAMPLE 18

The S. aureus strain is tested as described in Example 11, and a nutritional compound and device is prepared according to V26 of that example, with the difference that the three-dimensional structure has the shape of a 0.1 mm high and 60 cm diameter disc. A 3 m³ air volume is passed through the entire the volume, aided by an air filtration device that sucks it due to negative pressure. Contamination is simulated with the assay strain with a concentration of 10⁵ UFC/m³ prior to the air filtration, in order to verify if its flow is any influence on drying the structure or on its operation. When the device is exposed to air, it is moistened with the second solvent, consisting of distilled water and is left to incubate under the temperature and conditions described on Example 11. S. aureus can be identified after 240 minutes.

### EXAMPLE 19

Similar to Example 1, with the difference that two devices according to V1 of Example 1 are prepared, whereas to one device ciprofloxacin is added in the amount of 0.003 μg and in the other gentamicin in the amount of 0.2 μg. 0.2 ml of the microbial suspension is inoculated with an E. coli strain isolated from a urine culture with a concentration of 3×10⁸ UFC/ml and is incubated for 4 hours. No E. coli growth is observed after 4 hours in the device containing ciprofloxacin, or fluorescence in the device containing gentamicin.

1.-49. (canceled)

45. A method for the simultaneous detection, recovery, identification and counting of a plurality of microorganisms comprising, providing mixtures of nutrients specially selected from those that curtail the lag phase of growth in bacteria and moulds and which, together with florescent enzymatic, chromogenic or bioluminescent markers and other nutrient components or growth inhibitors, and embedding the mixtures in three-dimensional structures of natural or artificial clays or ceramics comprising cavities of different dimensions and forms and having specific surface areas of between 2×103 and 6×108 m²/m³.

46. A device for the simultaneous detection, recovery, identification and counting of a plurality of microorganisms comprising, an apparatus for providing mixtures of nutrients specially selected from those that curtail the lag phase of growth in bacteria and moulds and which, together with florescent enzymatic, chromogenic or bioluminescent markers and other nutrient components or growth inhibitors, further comprising a three-dimensional structure comprising natural or artificial clays or ceramics having cavities of different dimensions and forms having specific surface areas of between 2×103 and 6×108 m²/m³ with the mixtures embedded therein.

47. Method for detecting, recovering, identifying and/or simultaneously enumerating microorganisms, characterized by the fact that it dissolves or suspends a nutritional mixture that stimulates microbial growth in a solvent in quantities ranging from 1 to 50 g/L and one or multiple chromogenic, fluorogenic or bioluminescent enzymatic markers dissolved in a solvent, that absorbs the aforementioned components on one or multiple natural or artificial clay or ceramics three-dimensional structures; that it eliminates the solvent; that it puts the microbial cells or the samples that contain it into contact with the three-dimensional structure in the presence of a second solvent; that it maintains the structures in conditions that ensure the growth and identification of the variety of microorganisms due to the decay of the markers inside the nano-, micro- and macrocavities and over the surface of the structures, keeping them at temperature between 20 and 50° C. for a period that coincides with the longest duration of the lag phase and the final growth acceleration phase of the microorganism with the slowest development in order to detect, identify and enumerate the variety of microorganisms.

48. Method according to claim 47, characterized by the fact that a nutritional mixture selected among hydrolyzed enzymes of Spirulina platensis algae; extract of Saccharomyces cerevisiae obtained through enzyme hydrolysis and hydrolyzed enzyme of Torula fodder yeast; extract of sweet potatoes; tomato extract; hydrolyzed enzymes of beef heart tissue, of bovine blood and of beef liver; hydrolyzed enzymes of lactoalbumin from rennet whey, hydrolyzed enzymes or casein acids from buttermilk, and hydrolyzed or autolyzed from Eudrillus eugeniae.

49. Method according to claim 47, characterized by the fact that three-dimensional structures of natural or artificial clays and ceramics, selected among kaolinite, halloysite, dickite, nacrite, chrysolite, antigorite, lizardite, vermiculite, mica, hectorite, saponite, hydrotalcite, muscovite, chlorite, diatomaceous earth, bentonites (montmorillonite, sauconite, beidellite, nontrolite), clinoptilotites, hydroxyapatites, zeolites and calcium phosphates, or their combinations with a specific surface of 2×10³ to 6×10⁸ m²/m³ formed by a variety of nano-, micro- or macrocavities or their combinations.

50. Method according to claim 47, characterized by the addition to the first solvent one or multiple chromogenic, fluorogenic or bioluminescent enzymatic markers in quantities ranging from 0.01 to 2 g/L.

51. Method according to claim 47, characterized by the addition to the nutritional mixture of other hydrolyzed enzymes, hydrolyzed chemicals or algae protein extracts, microorganisms, vegetable components, higher animal tissue and their combinations in quantities ranging from 1 to 10 g/L.

52. Method according to claim 47, characterized because the calcium phosphates are: metaphosphate [Ca(PO₃)₂], monohydrated monocalcium phosphate [Ca(H₂PO₄)₂H₂O], dihydrogen tetracalcium phosphate (Ca₄H₂P₆O₂₀), heptacalcium phosphate [Ca₇(P₅O₁₆)₂], calcium pyrophosphate (Ca₂P₂O₇ and Ca₂P₂O₇.2H₂O), dicalcium phosphate [CaHPO₄, CaHPO₄.2H₂O and Ca(H₂PO₄)₂], tricalcium [Ca₃(PO₄)₂], octacalcium phosphate [Ca₈H₂(PO₄)₆.5H₂O], calcium-deprived hydroxyapatite [Ca₁₀-x(HPO₄)x(PO₄)₆-x(OH)₂], hydroxyapatite [Ca₁₀(PO₄)₆(OH)₂], tetracalcium phosphate [Ca₄O(PO₄)₂], apatite [Ca₁₀(PO₄)₆(OH,F,Cl,Br)₂], carbonate apatite [Ca₅(PO₄,CO₃)₃(OH,F)] or a mixture of two or more of any of them.

53. Method according to claim 47, characterized by using as first solvent distilled water; or deionised water; or aqueous salt solutions such as those from sodium chloride, phosphates; alcohols and alcohol solutions such as basic fuchsine 10% p/v solution in ethyl alcohol, or other substances that increases the solubility of enzymatic markers or the permeability of microorganism cells, such as dimethyl sulfoxide.

54. Method according to claim 47, characterized by dissolving the nutritional compound, the enzymatic markers and all other components in the first solvent with a 1 to 150 g/L ratio.

55. Method according to claim 47, characterized the elimination of the first solvent by drying the three-dimensional structure at ambient temperature with forced air circulation by convection, or at a temperature of 25 to 110° C. under atmospheric pressure or below atmospheric pressure over a 30 minute to 3-hour period, by sublimation, or by aspersion drying at a temperature of 90 to 180° C.

56. Method according to claim 47, characterized by the fact that it has detection and quantification limit of less than 1 UFC/10 L for liquid samples, less than 1 UFC/250 g for solid samples or less than 1 UFC/10 m³ for air and a maximum limit of up to 10⁹ UFC/ml or 10⁹ UFC/g or 10³ UFC/m³.

57. Method according to claim 47, characterized by the fact that water or a solution of salts is used as second solvent for most bacteria and fungi, a hypotonic or isotonic solution for extremophiles and microorganisms that live on sea waters, as well as hypertonic solutions, or the sample itself.

58. Method according to claim 47, characterized by the fact that a variety of microorganisms or the sample that contains them are put into contact with the three-dimensional structure with a 0.05 to 13 ml/g, or 0.1 to 10 m³/g ratio.

59. Method according to claim 47, characterized by the fact that a variety of microbial cells that may be formed by the diversity of microorganisms to be detected, recovered, identified and/or enumerated and that belong to a species, a genre, a group or a combination of these, including nanobacteria, bacteria, mould and yeasts, spores, hyphae or other propagules to be identified or the samples that contain them are put into contact with the surface of one or multiple three-dimensional structures, passing it through them or down to a certain depth; whereas the cells or samples that contain them are in the form of a gaseous or liquid phase suspension, in the form of a gel or with semisolid or solid consistency, applied directly over the structure, or by means of an application device.

60. Method according to claim 47, characterized by the fact that the three-dimensional structure is maintained throughout the microorganism detection phase under atmospheres with oxygen tension that may vary, from aerobic conditions for aerobic microorganisms and facultative aerobes, up to the total absence of this element for anaerobes or facultative anaerobes.

61. Method according to claim 47, characterized by the fact that the variety of cells are identified by visual or automatic detection of fluorescence; by the color change of the three-dimensional structure or of its consistency, texture, shine, opacity, tonality, homogeneity or transparency; or through the changes of color, shine, tonality, transparency or fluorescence of the second solvent or of the sample; or through the appearance of bioluminescence, both inside the cavities and over the surface of the structure; or by observing morphologic structures; or through metabolic reactions on the three-dimensional structure, in the second solvent or in the sample; or through a combination of some or all of the aforementioned identification methods.

62. Method according to claim 47, characterized by the detection or determination of cell concentration on the sample through their visual or automatic enumeration over the surface of the three-dimensional structure, or by measuring the intensity of the fluorogenic, colorimetric or bioluminescent signal under ultraviolet, visible or infrared light, through electrical, thermal or magnetic signals, through pH changes, or by quantifying the emission or consumption of gases derived from the activity of microorganisms during the lag phase or during the growth acceleration period, such as carbon dioxide, oxygen, hydrogen sulphide, ammonia or hydrogen.

63. Method according to claim 47, characterized by the fact that the resistance or sensibility to antimicrobial agents, such as bactericides, bacteriostatics, fungicides and/or cleaning solutions can be determined by adding the nutritional compound and the enzymatic markers of those substances to the mixture, observing total or partial growth inhibition or deceleration, the extension of the lag phase, or through the absence of substrate decay reaction.

64. Method according to claim 47, characterized by selecting three-dimensional structures among those whose cavity or particles correspond to:
nanocavities or particles, with diameters or clearances of up to 200 nm for nano- and microbacteria;
nano- and submicrocavities with diameters or clearances of 5 nm a 1000 nm for bacteria of different sizes;
microcavities with diameters or clearances of 1 μm to 1000 μm for yeast bacteria and cells; micro- and macrocavities with diameters or clearances of more than 1 μm and up to 2 mm for bacteria, yeasts and filamentous fungi;
combinations with all cavity diameters or clearances, from nano- up to macro- of 2 mm for the variety of microorganisms.

65. Method according to claim 47, characterized for the use of the three-dimensional clay or ceramic structures described above that have isomorphic replacement of ions by cations or that were previously functionalized with different ions that work as enzyme catalyzers, such as Na, K, Ca, Mg, P, Fe, Zn, forming superficial layers or distributed throughout its entire structure.

66. Method according to claim 47, characterized by the fact that further substances that promote or inhibit the growth of microorganisms that belong to certain genres, species or groups of microorganisms can be added to the first or second solvent, in quantities ranging from 0.01 up to 40 g/L.

67. Method according to claim 47, characterized by the addition of select salts, resins, natural plant extracts, fatty acids, esters, bactericides, bacteriostatics, alcohols, substances with superficial activity or their mixtures to the nutritional compound in quantities ranging from 0.01 to 2 g/L to the first or second solvent, or antibiotics or antifungal agents in quantities ranging from 10 to 100 μg/L to the first or second solvent.

68. Method according to claim 47, characterized by the addition of certain substances that contribute to the fixation of the nutritional compound and the enzymatic markers selected among alginates, natural polysaccharides; pectin, chitin, gum Arabic and other gums, starches, dextran and carboxymethylcellulose and other polymer derivatives of them; carrageenan, agar, agarose and artificial polymer derivatives, derivatives of vinyl alcohol, polybutylene, polyethylene and polypropylene, polyvinylpyrrolidone in quantities ranging from 0.01 to 0.5 g/g to the three-dimensional structure.

69. Method according to claim 47, characterized by the addition of certain substances to the three-dimensional structure in order to increase its absorption capacity, such as activated carbon and cellulose in quantities ranging from 2 to 4 mg/g.

70. Method according to claim 47, characterized by the use of a three-dimensional structure capable of “swelling” when it absorbs the sample containing microorganisms, or the second solvent containing the microorganisms and increasing its volume.

71. Method according to claim 47, characterized by the fact that the three-dimensional structure may form films or layers 5 nm to 1 mm thick; or columns up to 10 cm high; or spheres or pearls with a 5 nm to 10 mm diameter; hexagons or cubes; or cylinders or tubes with a 5 nm to 10 cm diameter and 5 nm to 10 cm high; or fibres, networks; or adopting the shape of the container that holds them.

72. Method according to claim 47, characterized by the use of a three-dimensional structure that shows different zones, different porosities and different diameters or clearances of the nano-, micro- and macrocavities present through its volume, length or diameter, distributing said zones as a gradient or in differentiated zones.

73. Method according to claim 47, characterized by the fact that it uses a three-dimensional structure containing different nutritional compounds and enzymatic markers throughout its volume, length or diameter, whereas those compounds are distributed as gradients or differentiated zones.

74. Method according to claim 47, characterized by the fact that the three-dimensional structure is maintained over supports shaped as sheets, layers or cylinders that may be pervious or impervious to them; or surrounded by impervious materials on at least 90% of its surface.

75. Devices for executing the method described in claims 47 above, characterized by the fact that one or multiple three-dimensional structures are formed by natural or artificial clays or ceramics, selected among kaolinite, halloysite, dickite, nacrite, chrysolite, antigorite, lizardite, vermiculite, mica, hectorite, saponite, hydrotalcite, muscovite, chlorite, diatomaceous earth, bentonites (montmorillonite, sauconite, beidellite, nontrolite), clinoptilotites, hydroxyapatites, zeolites and calcium phosphates, or their combinations with a specific surface of 2×10³ to 6×10⁸ m²/m³ in a variety of nano-, micro- or macrocavities or their combinations, and which contains inside of it or on its surface a nutritional mixture selected among the hydrolyzed enzymes of Spirulina platensis algae; extracts of Saccharomyces cerevisiae obtained through enzyme hydrolysis and hydrolyzed enzymes of Torula fodder yeast; extract of sweet potatoes, tomato extract; hydrolyzed enzymatic of beef heart tissue, of bovine blood and of beef liver; hydrolyzed enzymes of lactoalbumin from rennet whey, hydrolyzed enzymes or casein acids from buttermilk; and hydrolyzed or autolyzed from Eudrillus eugeniae and their combinations and one or multiple chromogenic, fluorogenic or bioluminescent enzymatic markers.

76. Devices according to claim 75, characterized by the fact that they contain one or multiple chromogenic, fluorogenic or bioluminescent enzymatic markers in quantities ranging from 0.0033 to 0.66 mg/g of three-dimensional structure.

77. Devices according to claim 75, characterized by the fact that they contain other hydrolyzed enzymes, hydrolyzed chemicals or algae protein extracts, microorganisms, vegetable components, higher animal tissue and their combinations in quantities ranging from up to 0.33 to 4 mg/g of three-dimensional structure.

78. Devices according to claim 75, characterized by the fact that they contain a mixture of nutritional compounds, enzymatic markers and other selective ingredients, inhibitors or growth promoters in quantities ranging from 0.33 mg/g up to 60 mg/g of three-dimensional structure.

79. Devices according to claim 75, characterized by the fact that the three-dimensional structure is formed by clays or ceramics with isomorphic replacement of ions with cations or previously functionalized with different ions that serve as enzyme catalyzers, such as Na, K, Ca, Mg, P, Fe, Zn, forming superficial layers or distributed throughout its entire structure.

80. Devices according to claim 75, characterized by the fact that the calcium phosphates that form its three-dimensional structure are selected among:
metaphosphate [Ca(PO₃)₂], monohydrated monocalcium phosphate [Ca(H₂PO₄)₂H₂O], dihydrogen tetracalcium phosphate (Ca₄H₂P₆O₂₀), heptacalcium phosphate [Ca₇(P₅O₁₆)₂], calcium pyrophosphate (Ca₂P₂O₇ and Ca₂P₂O₇.2H₂O), dicalcium phosphate [CaHPO₄, CaHPO₄.2H₂O and Ca(H₂PO₄)₂], tricalcium [Ca₃(PO₄)₂], octacalcium phosphate [Ca₈H₂(PO₄)₆.5H₂O], calcium-deprived hydroxyapatite [Ca₁₀-x(HPO₄)x(PO₄)₆-x(OH)₂-x], hydroxyapatite [Ca₁₄(PO₄)₆(OH)₂], tetracalcium phosphate [Ca₄₀(PO₄)₂], apatite [Ca₁₀(PO₄)₆(OH,F,Cl,Br)₂], carbonate apatite [Ca₅(PO₄,CO₃)₃(OH,F)] or indistinctly any combination of any of them.

81. Devices according to claim 75, characterized for having a detection and quantification limit of less than 1 UFC/10 L for liquid samples, less than 1 UFC/250 g for solid samples or less than 1 UFC/10 m³ of air and a maximum limit of up to 10⁹ UFC/ml or 10⁹ UFC/g or 10³ UFC/m³.

82. Devices according to claim 75, characterized by the fact that three-dimensional structures show cavities in the form of pores, channels, tubes, regular or irregular bags of different geometric shapes or their combinations; or if available as layers or sheets.

83. Devices according to claim 75, characterized by the fact that the three-dimensional structures are selected among those where the dimensions of their cavities or particles correspond to:
those of nanocavities or particles, preferably of shrivelled surfaces, with diameters or clearances of up to 200 nm for nano- and microbacteria;
nano- and submicrocavities with diameters or clearances of 5 nm to 1000 nm for bacteria of different sizes;
microcavities with diameters or clearances of 1 μm to 1000 μm for yeast bacteria and cells;
micro- and macrocavities with diameters or clearances of more than 1 μm and up to 2 mm for bacteria, yeasts and filamentous fungi;
combinations with all diameters or clearances of cavities from nano- up to macro- of 2 mm for the variety of microorganisms.

84. Devices according to claim 75, characterized by the fact that they contain selective microbial growth agents selected among salts, resins, natural plant extracts, fatty acids, esters, bactericides, bacteriostatics, alcohols, substances with superficial activity or mixtures in quantities ranging from 0.0033 a 0.8 mg/g of three-dimensional clay or ceramics structures and antibiotics or antifungal in quantities ranging from 0.033 to 0.33 μg/g.

85. Devices according to claim 75, characterized by the fact that they contain substances that contribute to the fixation of the nutritional compound and the enzymatic markers selected among alginates, natural polysaccharides; pectin, chitin, gum Arabic and other gums, starches, dextran and carboxymethylcellulose and other polymer derivatives of them; carrageenan, agar, agarose and artificial polymer derivatives, derivatives of vinyl alcohol, polybutylene, polyethylene and polypropylene, polyvinylpyrrolidone in quantities ranging from 0.01 to 0.5 gig of three-dimensional structure.

86. Devices according to claim 75, characterized by the fact that they contain substances that increase their absorption capacity, such as activated carbon and cellulose in the amount of 2 to 4 mg/g.

87. Devices according to claim 75, characterized by the fact that they films or layers 5 nm to 1 mm thick, or columns up to 10 cm high; or spheres or pearls with a 5 nm to 10 mm diameter; hexagons or cubes; or cylinders or tubes with a 5 nm to 10 cm diameter and 5 nm to 10 cm high; or fibres, networks; or adopting the shape of the container that holds them.

88. Devices according to claim 75, characterized by the fact that they may exhibit multiple zones with different porosities, diameters or clearances of nano-, micro- and macrocavities throughout their volume, length or diameter, distributing these zones as a gradient or in differentiated zones.

89. Devices according to claim 75, characterized by the fact that they contain different nutritional compounds and enzymatic markers throughout their volume, length or diameter, whereas those compounds are distributed as gradients or in differentiated zones.

90. Devices according to claim 75, characterized by the fact that they maintain the three-dimensional structure over supports shaped as sheets, layers or cylinders that may be pervious or impervious to them; or surrounded by impervious materials over at least 90% of their surface.
